(19) United States
(12) Patent Application Publication
(10) Pub. No.: US 2005/0244819 A1
(43) Pub. Date: Nov. 3, 2005

(54) Assay system for screening protease inhibitors

(75) Inventors: Ting-Jen Cheng, Claremont, CA (US); Chen-Chen Kan, Claremont, CA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Keck Graduate Institute, Claremont, CA (US)

(21) Appl. No.: 11/082,251

(22) Filed: Mar. 15, 2005

Related U.S. Application Data
(60) Provisional application No. 60/553,263, filed on Mar. 15, 2004.

(51) Int. Cl. C12Q 1/70; C12N 15/867; C12N 9/72; C07H 21/02

(52) U.S. Cl. 435/5; 435/69.3; 435/215; 435/456; 536/23.72

(57) ABSTRACT

Compositions and methods for identifying agents that inhibit protease activity are provided. In particular, polynucleotides, recombinant expression vectors, and host cells are provided that may be used in a bacterial cell-based assay for identifying agents that are inhibitors of protease activity, such as inhibitors of HIV protease activity. The bacterial cells express a precursor of a protease and encode a reporter polypeptide that contains a protease recognition sequence, which can be cleaved by the mature, catalytically active protease such that the reporter activity of the reporter polypeptide is decreased or eliminated.

### CROSS-REFERENCE TO RELATED APPLICATION

This application claims the benefit under 35 U.S.C. § 119(e) of U.S. Provisional Patent Application No. 60/553,263 filed Mar. 15, 2004, which is incorporated herein by reference in its entirety.

### STATEMENT OF GOVERNMENT INTEREST

This invention was made with government support under Grant No. CA 99898 awarded by the National Institutes of Health. The government may have certain rights in this invention.

### BACKGROUND OF THE INVENTION

1. Field of the Invention

The invention relates generally to compositions and methods useful for identifying agents that inhibit protease activity. In specific embodiments, the invention relates to compositions and methods for identifying agents that inhibit a human immunodeficiency virus (HIV) protease and for determining HIV protease inhibitor sensitivity profiles of HIV isolates.

2. Description of the Related Art

Human immunodeficiency virus (HIV) protease, which is essential for processing HIV viral polyproteins into individual structural proteins and replication enzymes during virus maturation, has been an attractive target for antiviral therapy development (Frankel et al., Annu. Rev. Biochem. 67:1-25 (1998)). Several HIV protease inhibitors designed to bind the active site of HIV protease have in vivo efficacy and are currently in clinical use. This line of therapy, by itself or in combination with reverse-transcriptase inhibitors, has revolutionized antiviral treatments and dramatically lowered the number of deaths due to acquired immune deficiency syndrome (AIDS) (Wlodawer et al., Annu. Rev. Biochem. 62:543-85 (1993)). However, widespread use of HIV protease inhibitors has caused the rapid emergence of drug-resistant HIV proteases (J. W. Erickson, HIV-1 Protease as a Target for AIDS Therapy in PROTEASE INHIBITORS IN AIDS THERAPY 1-25 (R. C. Ogden et al., eds. 2001), rendering AIDS with no definitive cure. In these HIV proteases, mutations have been found in forty-nine of the ninety-nine amino acids of the coding sequence. Substitutions at eighteen or more positions are directly correlated with loss of responsiveness to HIV protease inhibitor treatment. Because the existing HIV protease inhibitors target the active site of HIV protease and have similar structures, most of the drug-resistant HIV proteases confer cross-resistance to multiple HIV proteases.

Accordingly, a great need exists for second-generation HIV protease inhibitors with different chemical structures and/or with an alternative mode of inhibition, such as targeting activation of the protease, that is, dimerization and folding of the HIV protease.

### BRIEF SUMMARY OF THE INVENTION

Provided herein are compositions and methods for identifying agents that inhibit protease activity. In one embodiment, the invention provides a polynucleotide comprising a nucleotide sequence that encodes a precursor of a protease and a reporter polypeptide, wherein the reporter polypeptide comprises a protease recognition sequence. In one embodiment, the precursor of the protease comprises a human immunodeficiency virus (HIV) transframe region and the protease. In certain embodiments, the transframe region is fused to or linked to the amino terminal end of the protease domain. In another embodiment, the precursor of the protease that is encoded by the polynucleotide is autoproteolytically processed to yield the protease. In one embodiment, the protease is an aspartyl protease and in certain embodiments, the protease is a viral protease. In certain specific embodiments, the viral protease is a human immunodeficiency virus (HIV) protease. In a particular embodiment, the precursor of the protease comprises a human immunodeficiency virus (HIV) transframe region and wherein the protease is an HIV protease. In a specific embodiment, the HIV protease is an HIV protease mutant. In certain embodiments, the reporter polypeptide is β-galactosidase, and in certain specific embodiments, the encoded protease recognition sequence comprises the amino acid sequence VSFNFPQITL (SEQ ID NO: 1). In one embodiment, the polynucleotide is a 2-cistron construct, wherein a first cistron encodes the precursor of a protease and a second cistron encodes the reporter polypeptide. In another embodiment, the protease recognition sequence is inserted at a position in the reporter polypeptide such that in the absence of cleavage of the reporter polypeptide by the protease, the reporter polypeptide has reporter activity that is comparable to reporter activity of the wild type reporter polypeptide, and such that in the presence of cleavage by the protease, the reporter polypeptide has decreased reporter activity compared with the wild type reporter polypeptide.

Also as described herein, the invention provides a recombinant expression vector comprising at least one promoter operatively linked to a polynucleotide comprising a nucleotide sequence that encodes a precursor of a protease and a reporter polypeptide, wherein the reporter polypeptide comprises a protease recognition sequence. The invention also provides a host cell that that comprises the recombinant expression vector. In certain embodiments, the host cell is a bacterial cell and in particular embodiments, the bacterial cell is an E. coli cell. In one embodiment, the precursor of the protease comprises a human immunodeficiency virus (HIV) transframe region and the protease. In certain embodiments, the transframe region is fused to or linked to the amino terminal end of the protease domain. In another embodiment, the precursor of the protease that is encoded by the polynucleotide is autoproteolytically processed to yield the protease. In one embodiment, the protease is an aspartyl protease and in certain embodiments, the protease is a viral protease. In certain specific embodiments, the viral protease is a human immunodeficiency virus (HIV) protease. In another specific embodiment, the HIV protease is an HIV protease mutant. In a particular embodiment, the precursor of the protease comprises a humani immunodeficiency virus (HIV) transframe region and wherein the protease is an HIV protease. In certain embodiments, the reporter polypeptide is β-galactosidase, and in certain specific embodiments, the encoded protease recognition sequence comprises the amino acid sequence VSFNFPQITL (SEQ ID NO: 1). In one embodiment, the polynucleotide is a 2-cistron construct, wherein a first cistron encodes the precursor of a protease and a second cistron encodes the reporter polypeptide. In another embodiment, the protease recognition sequence is inserted at a position in the reporter polypeptide such that in the absence of cleavage of the reporter polypeptide by the protease, the reporter polypeptide has reporter activity that is comparable to reporter activity of the wild type reporter polypeptide, and such that in the presence of cleavage by the protease, the reporter polypeptide has decreased reporter activity compared with the wild type reporter polypeptide.

The invention also provides a method for identifying an agent that inhibits catalytic activity of a protease comprising (a) contacting a candidate agent with a cell that expresses a precursor of a protease and that expresses a reporter polypeptide, wherein the reporter polypeptide comprises a protease recognition sequence, under conditions and for a time that permit expression and autoproteolytic processing of the precursor of the protease and cleavage of the reporter polypeptide by the protease; (b) detecting a reporter activity of the reporter polypeptide; and (c) comparing the level of reporter activity of the reporter polypeptide in the presence and absence of the candidate agent, wherein an increase in the level of reporter activity of the reporter polypeptide in the presence of the candidate agent compared with the level of reporter activity of thereporter polypeptide in the absence of the candidateagent indicates that the candidate agent inhibits the protease. In one embodiment, the precursor of the protease comprises a human immunodeficiency virus (HIV) transframe region and the protease. In certain embodiments, the transframe region is fused to or linked to the amino terminal end of the protease domain. In another embodiment, the precursor of the protease that is encoded by the polynucleotide is autoproteolytically processed to yield the protease. In one embodiment, the protease is an aspartyl protease and in certain embodiments, the protease is a viral protease. In certain specific embodiments, the viral protease is a human immunodeficiency virus (HIV) protease. In another specific embodiment, the HIV protease is an HIV protease mutant. In a particular embodiment, the precursor of the protease comprises a human immunodeficiency virus (HIV) transframe region and wherein the protease is an HIV protease. In certain embodiments, the reporter polypeptide is β-galactosidase, and in certain specific embodiments, the encoded protease recognition sequence comprises the amino acid sequence VSFNFPQITL (SEQ ID NO: 1). In one embodiment, the polynucleotide is a 2-cistron construct, wherein a first cistron encodes the precursor of a protease and a second cistron encodes the reporter polypeptide. In another embodiment, the protease recognition sequence is inserted at a position in the reporter polypeptide such that in the absence of cleavage of the reporter polypeptide by the protease, the reporter polypeptide has reporter activity that is comparable to reporter activity of the wild type reporter polypeptide, and such that in the presence of cleavage by the protease, the reporter polypeptide has decreased reporter activity compared with the wild type reporter polypeptide.

The invention also provides a method for identifying an agent that inhibits catalytic activity of a human immunodeficiency virus (HIV) protease comprising (a) contacting a candidate agent with a cell that expresses a precursor of an HIV protease and that expresses a reporter polypeptide, wherein the reporter polypeptide comprises an HIV protease recognition sequence, under conditions and for a time that permit expression and autoproteolytic processing of the precursor of the HIV protease and cleavage of the reporter polypeptide by the HIV protease; (b) detecting a reporter activity of the reporter polypeptide; and,(c) comparing a level of reporter activity of the reporter polypeptide in the presence and absence of the candidate agent; wherein an increase in the level of reporter activity of the reporter polypeptide in the presence of the candidate agent compared with a level of reporter activity of the reporter polypeptide in the absence of the candidate agent indicates that the candidate agent inhibits the HIV protease. In certain embodiments, the reporter polypeptide is β-galactosidase, and in particular embodiments, the HIV protease recognition sequence comprises the amino acid sequence VSFNFPQITL (SEQ ID NO: 1). In a specific embodiment, the HIV protease is an HIV protease mutant. In one embodiment, the precursor of the HIV protease comprises an HIV transframe region.

In a particular embodiment of this method, the host cell comprises a recombinant expression vector (construct) comprising at least one promoter operatively linked to a polynucleotide comprising a nucleotide sequence that encodes a precursor of an HIV protease and a reporter polypeptide, wherein the reporter polypeptide comprises an HIV protease recognition sequence. In one embodiment, the precursor of the HIV protease comprises a human immunodeficiency virus (HIV) transframe region and the HIV protease. In certain embodiments, the transframe region is fused to or linked to the amino terminal end of the protease domain. In another embodiment, the precursor of the HIV protease that is encoded by the polynucleotide is autoproteolytically processed to yield the HIV protease. As described above, in another specific embodiment, the HIV protease is an HIV protease mutant. In certain embodiments, the reporter polypeptide is β-galactosidase, and in certain specific embodiments, the encoded protease recognition sequence comprises the amino acid sequence VSFNFPQITL (SEQ ID NO: 1). In one embodiment, the polynucleotide is a 2-cistron construct, wherein a first cistron encodes the precursor of a protease and a second cistron encodes the reporter polypeptide. In another embodiment, the protease recognition sequence is inserted at a position in the reporter polypeptide such that in the absence of cleavage of the reporter polypeptide by the protease, the reporter polypeptide has reporter activity that is comparable to reporter activity of the wild type reporter polypeptide, and such that in the presence of cleavage by the protease, the reporter polypeptide has decreased reporter activity compared with the wild type reporter polypeptide.

The invention also provides a method for determining sensitivity of a human immunodeficiency virus (HIV) isolate to an inhibitor of HIV protease activity comprising (a) preparing a host cell that comprises a recombinant expression vector comprising a promoter operatively linked to (i) a nucleotide sequence that encodes an HIV transframe region fused to the nucleotide sequence encoding an HIV protease from the HIV isolate and (ii) a nucleotide sequence that encodes a reporter polypeptide comprising a protease recognition sequence; (b)contacting an inhibitor of HIV protease activity with the host cell, under conditions and for a time that permit expression and autoproteolytic processing of the precursor of the HIV protease and cleavage of the reporter polypeptide by the HIV protease; (c)detecting a reporter activity of the reporter polypeptide; and (d) comparing a level of reporter activity of the reporter polypeptide in the presence and absence of the inhibitor of HIV protease activity, wherein an alteration in the level of reporter activity of the reporter polypeptide in the presence of the inhibitor of HIV protease activity compared with the level of reporter activity of the reporter polypeptide in the absence of the inhibitor of HIV protease activity indicates the level of sensitivity of the HIV protease from the HIV isolate to the inhibitor of HIV protease activity. In a certain embodiment, the HIV isolate is obtained from a biological sample, and in another certain embodiment, the biological sample is obtained form a subject who is infected with the HIV isolate. In certain embodiments, the reporter polypeptide is β-galactosidase, and in certain specific embodiments, the encoded protease recognition sequence comprises the amino acid sequence VSFNFPQITL (SEQ ID NO: 1). In specific embodiments, the inhibitor of HIV protease activity is selected from amprenavir (Agenerase®); fosamprenavir (Lexiva®); indinavir (Crixivan®); nelfinavir (Viracept®); ritonavir (Norvir®); and saquinavir (Fortovase®).

The invention also provides an assay system comprising (a) a cell that comprises a recombinant expression vector comprising a promoter operatively linked to (i) a nucleotide sequence that encodes an HIV transframe region fused to the nucleotide sequence that encodes an HIV protease to provide a precursor of the HIV protease and (ii) a nucleotide sequence that encodes a reporter polypeptide comprising an HIV protease recognition sequence; (b) a compound that induces expression of the precursor of the HIV protease and the reporter polypeptide; and (c) a reporter polypeptide substrate. In certain embodiments, the host cell is a bacterial cell and in particular embodiments, the bacterial cell is an E. coli cell. In certain embodiments, the reporter polypeptide is β-galactosidase, and in certain specific embodiments, the encoded protease recognition sequence comprises the amino acid sequence VSFNFPQITL (SEQ ID NO: 1). In one embodiment, the reporter polypeptide substrate is o-nitrophenyl-β-D-galactoside (ONPG) or 6,8 difluoro-4-methylumbelliferyl β-d-galactopyranoside. In another embodiment the assay system further comprises an agent that inhibits HIV protease activity. In another specific embodiment, the assay system further comprises at least one candidate agent to be screened for its capability to inhibit HIV protease activity. In one embodiment, the protease recognition sequence is inserted at a position in the reporter polypeptide such that in the absence of cleavage of the reporter polypeptide by the protease, the reporter polypeptide has reporter activity that is comparable to reporter activity of the wild type reporter polypeptide, and such that in the presence of cleavage by the protease, the reporter polypeptide has decreased reporter activity compared with the wild type reporter polypeptide.

In another embodiment an assay system is provided that comprises (a) a cell that comprises a recombinant expression vector comprising a promoter operatively linked to (i) a nucleotide sequence that encodes a precursor of a protease and (ii) a nucleotide sequence that encodes a reporter polypeptide comprising a protease recognition sequence; (b) a compound that induces expression of the precursor of the protease and the reporter polypeptide; and (c) a reporter polypeptide substrate. In certain embodiments, the host cell is a bacterial cell and in particular embodiments, the bacterial cell is an E. coli cell. In certain embodiments, the reporter polypeptide is β-galactosidase, and in certain specific embodiments, the encoded protease recognition sequence comprises the amino acid sequence VSFNFPQITL (SEQ ID NO: 1). In one embodiment, the reporter polypeptide substrate is o-nitrophenyl-β-D-galactoside (ONPG) or 6,8 difluoro-4-methylumbelliferyl β-d-galactopyranoside. In another embodiment, the precursor of the protease comprises a human immunodeficiency virus (HIV) transframe region and the protease. In certain embodiments, the transframe region is fused to or linked to the amino terminal end of the protease domain. In another embodiment, the precursor of the protease that is encoded by the polynucleotide is autoproteolytically processed to yield the protease. In one embodiment, the protease is an aspartyl protease and in certain embodiments, the protease is a viral protease.

These and other embodiments of the invention will become evident upon reference to the following detailed description and attached drawings. In addition, all U.S. patents, U.S. patent application publications, U.S. patent applications, foreign patents, foreign patent applications, and non-patent publications referred to in this specification and/or listed in the Application Data Sheet, which describe in more detail certain embodiments of this invention, are incorporated by reference in their entireties.

### BRIEF DESCRIPTION OF THE DRAWINGS

FIG. 1 illustrates co-expression of HIV protease and β-galactosidase in E. coli. FIG. 1A presents a schematic of the two-cistron constructs described herein. The first cistron is the HIV protease precursor, comprising the transframe (TF), including the transframe peptide (TFP) and p6^{pol}, and the protease domain (PR^{WT}). The second cistron is the engineered β-galactosidase, containing an insertion of the protease cleavage site, Val-Ser-Phe-Asn-Phe-Pro-Gln-Ile-Thr-Leu (SEQ ID NO: 1), at the Saul site (amino acid 131) (β-Gal^{PR}). The inactive HIV protease in which the catalytic Asp25 was substituted with Asn mutation was used to prepare the pTF-PR^{D25N} construct. The arrows represent the cleavage sites of HIV protease. FIG. 1B presents an immnunoblot demonstrating auto-proteolytic processing of the HIV protease precursor. The HIV protease and precursor were detected with HIV-1 protease antiserum. Cells bearing individual plasmids (indicated at the top of the blots) were induced with 0.2% arabinose for 3 hr at 30° C. The cells were then collected and processed for separation on 4 to 12% Bis-Tris gels followed by immunoblot analysis. “M” denotes the molecular weight standards. Each lane contained the lysate from 0.3 OD₆₃₀ equivalents of cells, except the lane for PR^{D25N}, which contained 0.1 OD₆₃₀ equivalents of cells expressing PR^{D25N}. FIG. 1C illustrates cleavage of β-Gal^{PR} detected with antibodies against β-galactosidase. The plasmids present in each sample are indicated above the lanes. Each lane contained the lysate from 0.3 OD₆₃₀ equivalents of cells.

FIG. 2A illustrates the synthesis reaction used for preparation of a compound library. FIG. 2B presents thirty carboxylic acid structures that were substituted at the P2 position. FIG. 2C illustrates the structure of compound E2 and presents its Kᵢ and in vitro IC₅₀s against wildtype HIV protease and three mutant HIV proteases.

FIG. 3 illustrates immunoblot results demonstrating in vivo dose-dependent effects of protease inhibitors on auto-processing of the protease precursor, TF-PR^{WT}, in E. coli cells. Protease inhibitors were pepstatin A (Pep A) (FIG. 3A); APV (amprenavir) (FIG. 3B); IDV (indinavir) (FIG. 3C); NFV (nelfinavir )(FIG. 3D); RTV (ritonavir) (FIG. 3E); and SQV (saquinavir) (FIG. 3F). The E. coli cells containing the expression construct pPR^{D25N}, pTF-PR^{WT}, or pTF-PR^{D25N} were induced with 0.2% arabinose for 3 hours at 30° C. in the presence of 2% DMSO. Simultaneously, the E. coli cells expressing TF-PR^{WT} were treated with the HIV protease inhibitors at the indicated concentrations in 2% DMSO. The cells were then collected, and the proteins were analyzed by Western blotting with HIV- 1 antiserum. Each lane contained 0.3 OD₆₃₀ equivalents of cells, except the lane for PR^{D25N}, which contained 0.1 OD₆₃₀ equivalents of cells expressing PR^{D25N}.

FIG. 4 illustrates in vivo dose-dependent effects of HIV protease inhibitors against the wild-type HIV protease. The in vivo activity of the HIV protease inhibitors was determined by the level of inhibition of the cleavage-induced loss of β-galactosidase activity. The cells coexpressing TF-PR^{WT} and β-Gal_{PR} were treated with HIV protease inhibitors at the indicated concentrations. Subsequently, β-galactosidase activities (micromoles perminute per milligram of total protein) in the treated E. coli cells were determined. Representative results from three separate experiments are shown, and each experiment had a triplicate set of each sample.

FIG. 5 presents data demonstrating in vivo dose-dependent effects of HIV protease inhibitors against the D30N drug-resistant HIV protease mutant. The in vivo activities of the HIV protease inhibitors were determined as described in the legend to FIG. 4 using E. coli co-expressing TF-PR^{D30N} and β-Gal^{PR}. β-galactosidase activities (micromoles per minute per milligram of total protein) in the treated E. coli cells were determined, and the data were used to generate the dose-response curves by curve-fitting with MATLAB. Representative results from three separate experiments are shown, and each experiment had a triplicate set of each sample.

FIG. 6 presents data showing in vivo dose-dependent effects of compound E2 against HIV proteases in the E. coli-based screening system. FIG. 6A: Inhibition of auto-processing of the wild-type HIV protease precursor. The compound E2 was added to E. coli cells containing the expression construct, pTF-PR^{WT}, and Western blot analysis with HIV protease antiserum was performed. Each lane contained 0.3 OD₆₃₀ equivalents of cells, except the lane for PR^{D25N}, which contained 0.1 OD₆₃₀ equivalents of cells expressing PR^{D25N}. FIG. 6B: Inhibition of cleavage-induced loss of β-galactosidase activity in the presence of the wild-type HIV protease. The E2-treated cells were processed to detect β-galactosidase activity by using whole bacterial cells plated in 96-well microplates. FIG. 6C and 6D present data demonstrating in vivo dose-dependent effects of APV (FIG. 6C) and E2 (FIG. 6D) against the I84V HIV protease mutant. The in vivo activity of the HIV protease inhibitors was determined by measuring inhibition of the cleavage-induced loss of β-galactosidase activity. The cells co-expressing TF-PR^{I84V} and β-Gal^{PR} were treated with HIV protease inhibitors at the indicated concentrations. β-Galactosidase activities (micromoles per minute per milligram of total protein) in the treated E. coli cells were determined, and the data were used to generate the dose-response curves by curve-fitting with MATLAB. Representative results from three separate experiments are shown, and each experiment had a triplicate set of each sample.

### DETAILED DESCRIPTION OF THE INVENTION

New human immunodeficiency virus (HIV) protease inhibitors are urgently needed for combating the drug-resistance problem in the battle against acquired immune deficiency syndrome (AIDS). The present invention relates to the discovery, as described herein, of a safe, convenient, and cost-effective E. coli-based assay system that facilitates lead discovery of protease inhibitors, including HIV protease inhibitors. In one embodiment, the E. coli-based system comprises co-expression of an engineered β-galactosidase, which serves as an HIV protease substrate, and the HIV protease precursor, which comprises the transframe region and the protease domain. Autoproteolytic processing (auto-processing) of the HIV protease precursor releases the mature, catalytically active HIV protease. Subsequently, the HIV protease cleaves β-galactosidase, resulting in a loss of the β-galactosidase activity, which can be detected in an assay system, including a high-throughput screen. The invention relates in part to the surprising discovery that expression of the protease precursor instead of the mature, catalytically active protease allows detection of reporter polypeptide activity. In a bacterial cell, when the catalytically active HIV protease is encoded by a polynucleotide (that is, the polynucleotide encodes only the mature HIV protease and not a precursor of the HIV protease), expression of the HIV protease results in bacterial cell death before reporter polypeptide activity can be detected.

As described herein, this E. coli-based system may be used to identify inhibitors that possess inhibitory activity against HIV protease and that may also have solubility/permeability properties that are favorable for activity in vivo. The compositions and methods described herein can be also used to generate drug-resistance profiles of HIV strains and thus may be used to indicate therapeutic uses of HIV protease inhibitors for treating subjects infected with drug-resistant HIV strains.

The methods described herein represent a screening method that may be used for high-throughput identification of agents that target proteases, including aspartate proteases such as the HIV protease, or variants or mutants thereof. The bacterial assay system comprises co-expression of an engineered reporter polypeptide, such as a modified β-galactosidase, which serves as a protease substrate, and a precursor of a protease, which lacks proteolytic activity. The protease precursor comprises an amino acid sequence (also referred to as a precursor domain), generally at the amino terminal end, that is cleaved (hydrolysis of a peptide bond between adjacent amino acids) from the precursor protease to yield the mature, catalytically active protease. The precursor protease may be cleaved by autoproteolysis or may be cleaved by a different protease.

The engineered reporter polypeptide includes a protease recognition sequence, that is, an amino acid sequence that is recognized by the protease of interest and that is either coincident with or near to the amino acid sequence susceptible to cleavage (proteolytic hydrolysis) by the protease. The protease recognition sequence may be introduced by recombinant molecular biology methods as described herein and with which persons skilled in the art are familiar. Alternatively, the amino acid sequence of a wildtype reporter polypeptide may have a protease recognition sequence that is recognized by a protease of interest. A protease recognition sequence refers to a consecutive amino acid sequence that is recognized and required for proteolytic cleavage by a protease of interest. A protease recognition sequence may be coincident with the protease cleavage site (i.e., the site at which the cleavage by the protease occurs). In other words, the protease recognition sequence may include one or more amino acids on either side of the peptide bond to be hydrolyzed by the protease. Alternatively, the protease recognition sequence may be one, two, or more amino acids distal, toward the amino or carboxy terminus, to the cleavage site of the protease. Accordingly, the protease cleaves the polypeptide comprising the protease recognition sequence at or near the protease recognition sequence. Thus the reporter polypeptide comprises a protease cleavage site that is susceptible to proteolytic cleavage by the protease of interest. In certain embodiments, a protease recognition sequence is derived from the amino acid sequence of a naturally occurring substrate of the protease of interest.

The released, catalytically active protease then may recognize the protease recognition sequence present in the reporter polypeptide, which reporter polypeptide then serves as a substrate for the protease of interest. In a preferred embodiment, the recognition sequence is present within the reporter polypeptide at a location that does not alter the reporter activity unless the reporter polypeptide is cleaved by the protease. Thus, the level of protease activity or inhibition of protease activity correlates with the reporter activity, and determination of reporter activity indicates the capability of a compound or agent to inhibit the proteolytic activity of the protease of interest.

A reporter polypeptide refers to a polypeptide that when it is expressed in a cell, its presence can easily be measured, for example, by detection of a catalytic activity if the reporter polypeptide is an enzyme. Such catalytic activity is preferably readily detected and can be distinguished from the background activity of other proteins in a cell. Reporter polypeptides include, for example, chloramphenicol acetyltransferase (CAT), luciferase, and β-galactosidase. Other reporter polypeptides include but are not limited to alkaline phosphatase, β-glucuronidase, green fluorescent protein, red fluorescent protein, aequorin, and horseradish peroxidase.

As described herein, a reporter polypeptide that tolerates insertion of amino acids without adversely affecting or significantly altering the reporter activity is useful for the systems and methods described herein. Such a reporter polypeptide includes β-galactosidase (see Sambrook et al., 17.97 Molecular Cloning: A Laboratory Manual (3rd ed. 2001). E. coli β-galactosidase (molecular weight=465,412; EC 3.2.1.23) is a tetramer of four identical polypeptide subunits that each consist of 1023 amino acids, the sequence of which has been long known in the art (see Fowler and Zabin, J. Biol. Chem. 253:5521-25 (1978)). The enzyme is encoded by the lacZ gene of the lac operon (see Kalnins et al., EMBO J 2:593-97 (1983); see, e.g., GenBank Accession No. V00296). β-galactosidase hydrolyzes β-D-galactopryanosides and is essential for the hydrolysis in E. coli of the disaccharide lactose (serving as a carbon source) into glucose and galactose. The enzyme can also catalyze hydrolysis of synthetic analogs of lactose, including chromogenic substrates such as o-nitrophenyl-β-D-galactoside (ONPG) and 5-bromo-4-chloro-3-3indolyl-β-D-galactoside (X-gal), and also can hydrolyze fluorogenic substrates, for example, 4-methylumbelliferyl-β-D-galactoside and 6,8 difluoro-4-methylumbelliferyl β-d-galactopyranoside (DifNuG) and other substrates used by persons skilled in the art (see, e.g., Rothstein et al., Proc. Nat'l Acad. Sci. USA 77: 7372-76 (1980); Miller, Experiments in Molecular Genetics (Cold Spring Harbor, N.Y. 1972); Roth, Methods Biochem. Anal. 17: 189-285 (1969); Youngman, in Plasmids: A practical approach (K. G. Hardy, ed.), pp. 79-103 (IRL Press, Oxford, United Kingdom 1987). In addition, by using β-galactosidase containing a recognition sequence of a protease of interest as a reporter in a bacterial system, the activity of the reporter may be determined by plating the bacteria cells that express the polynucleotide encoding the reporter on a X-Gal-containing agar medium plate. If the β-galatosidase is catalytically active, the colonies of the bacterial cells are blue. Conversely, if the β-galactosidase is catalytically inactive, the bacterial colonies are white.

In one embodiment of the invention, the protease of interest is a protease associated with a microorganism that is an infectious disease organism, such as for example, a protease called lethal factor from Bacillus anthracis or a viral protease (e.g., poliovirus; HIV (HIV-1 and HIV-2); human T-cell lymphotropic viruses (HTLV); murine mammary tumor virus (MMTV)). HIV and MMTV proteases are examples of proteases that belong to the class of aspartyl proteases. Other exemplary aspartyl proteases include pepsin, cathepsin D, and retin, gastricsin, napsin, cathepsin E, BACE 1 and 2. Because proteases that are associated with infectious disease organisms are often involved in pathogenesis of the disease, identifying inhibitors of such proteases is useful for treating subjects who have the infectious disease or who are susceptible to acquiring or manifesting symptoms of the infectious disease. In a particular embodiment, the compositions and methods of an E. coli-based system as described herein may be used to identify inhibitors that possess inhibitory activity against HIV protease.

The protease precursor polypeptide and the reporter polypeptide are expressed in a host cell under conditions and for a time sufficient that allow expression of these polypeptides, which conditions (e.g., temperature, growth media, addition of compounds that induce expression) and time may be determined according to standard methods routinely practiced by persons skilled in the art. The expressed protease precursor undergoes autoproteolytic cleavage to yield the catalytically active protease, which then cleaves the reporter polypeptide at or near the protease recognition sequence within the reporter polypeptide sequence. Cleavage of the reporter polypeptide alters the reporter activity of the polypeptide, which can be determined according to methods appropriate for the particular reporter activity. In particular embodiments, alteration of the reporter activity is a significant decrease (i.e., a statistically significant decrease) or a complete reduction of activity compared with the activity of the non-proteolytically cleaved reporter polypeptide. When the host cell is contacted with an inhibitor of the protease encoded by the polynucleotide, and the inhibitor is able to diffuse or be transported (or in some manner pass through the cell membrane) into the cell, the extent to which protease activity is inhibited, which correlates with the. extent to which the reporter polypeptide is cleaved, can be assessed by determining the level of reporter activity.

In one embodiment, the protease of interest is an HIV protease. By way of background, HIV protease is an aspartyl protease. The active HIV protease is an obligatory dimer, consisting of two identical and noncovalently associated monomers. The active siteof the enzymre is formed at the dimer interface and contains two conserved catalytic aspartic acid residues, one from each monomer (Cheng et al., Proc., Nat'l Acad. Sci. USA 87:9660-64 (1990)). Instead of being translated as an active enzyme, HIV protease is synthesized as an integral part of the viral Gag-Pol polyprotein and needs to be activated to exhibit catalytic activity. Activation of the HIV protease requires appropriate folding and dimerization of the protease domain in the viral Gag-Pol polypeptide to form an active site, which then can catalyze the hydrolysis of peptide bonds at cleavage sites to release the mature HIV protease (Zybarth et al., J. Virol. 69:3878-84 (1995)).

Not wishing to be bound by theory, the molecular mechanism of HIV protease activation may involve regulation of HIV protease activity by the transframe region (TF) that flanks the N terminus of the protease domain in the Pol gene (Pettit et al., J. Virol. 77:366-74 (2003)). The native TF comprises an 8-amino-acid transframe peptide and a 48-amino-acid p6^{pol} protein. Purified recombinant p6^{pol} protein was shown to have inhibitory effects on HIV protease activity (Paulus et al., J. Biol. Chem. 274:21539-43 (1999); see also, e.g., GenBank Accession No. NP_{—}787043). The intact HIV protease precursor, including TF and the protease domain, has a very low dimer stability relative to that of the mature enzyme and exhibits a negligible level of stable tertiary structure (Wondrak et al., Biochemistry 35:12957-62 (1996)). Louis. et al. (Nat. Struct. Biol. 6:868-75 (1999)) proposed that TF interacts with the dimer interface and thus destabilizes the dimeric structure to regulate HIV protease activity. The solution structure of the HIV protease precursor confirmed that TF hinders dimerization of the HIV protease domain and thus alters the catalytic activity of HIV protease (Ishima et al., J. Biol. Chem. 278:43311-19(2003)).

Standard screening methods for identifying HIV protease inhibitors include both in vitro and in vivo assays. While the in vitro enzyme kinetic assays incorporate purified recombinant HIV proteases and specific substrates, the in vivo assays use mammalian cells and the HIV viruses to evaluate the permeability as well as the in vivo antiviral activities of the potential drug leads. These current protocols, however, are complex and time-consuming. The use of an E. coli-based system as described herein is a simple and safe alternative method for HIV protease inhibitor screening.

Polynucleotides, Vectors, and Host Cells

A polynucleotide that encodes the protease or a precursor of the protease and a polynucleotide (which may be the same polynucleotide or a different polynucleotide) that expresses a reporter polypeptide comprising the recognition sequence of the protease are transfected or transformed into a host cell, such as a bacterial cell (e.g., E. coli, Salmonella typhimurium, etc.). Polynucleotides as described herein may be single-stranded (coding or antisense) or double-stranded, and may be DNA (genomic, cDNA, or synthetic) or RNA molecules.

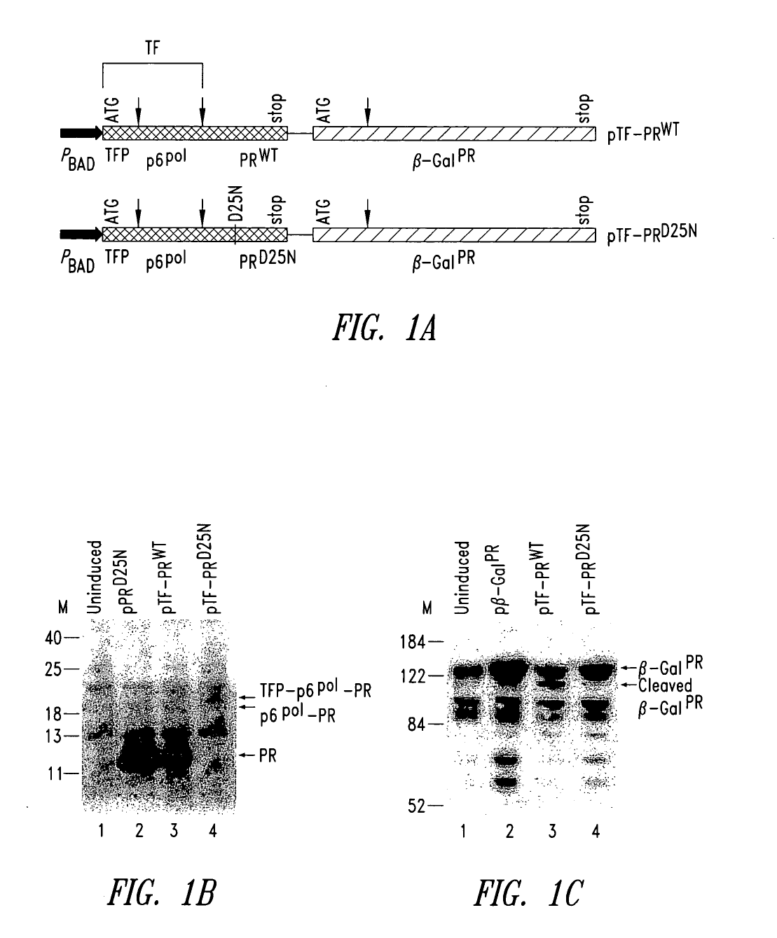

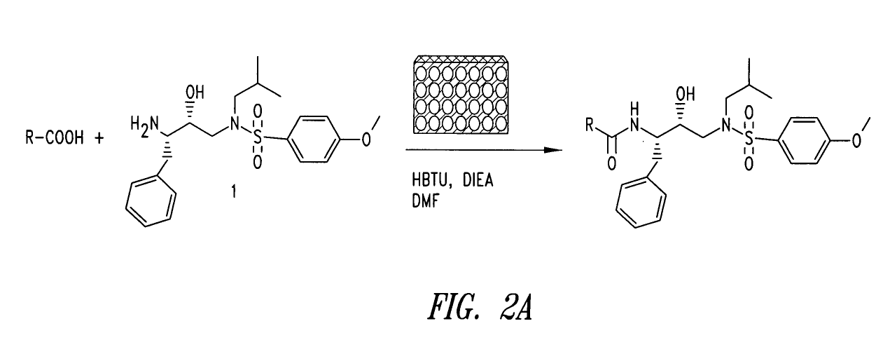

In one embodiment, a polynucleotide comprises a nucleotide sequence that encodes a protease precursor that is a precursor of the HIV protease and comprises a nucleotide sequence that encodes a reporter polypeptide that is β-galactosidase, into which is inserted a HIV protease recognition amino acid sequence. In a certain embodiment, the nucleotide sequence encoding the HIV protease precursor and the nucleotide sequence encoding β-galactosidase comprising the HIV protease recognition sequence are included within the same polynucleotide. By way of example, as illustrated in FIG. 1A, the polynucleotide is a two-cistron expression cassette comprising a single promoter operatively linked to a nucleotide sequence encoding the HIV precursor polypeptide and to a nucleotide sequence encoding β-galactosidase comprising the HIV protease recognition sequence.

The HIV protease precursor (also referred to herein as TF-PR) comprises a transframe (TF) region and the HIV protease domain. The TF region of the natural TF-PR is immediately adjacent to the amino terminal end of the HIV protease domain. Accordingly, a polynucleotide comprises a nucleotide sequence that encodes the transframe region, which is fused to or linked to, and in frame with, (immediately upstream of the 5′ end of the nucleotide sequence encoding the mature HIV protease) a nucleotide sequence that encodes the HIV protease domain. In certain embodiments, the TF region comprises 56 amino acids immediately adjacent to the amino terminal end of a mature HIV protease (i. e., a phenylalanine residue at the carboxy terminus of the transframe region is immediately adjacent to the amino terminalto the proline at the amino terminus of the mature HIV protease). Autoproteolytic processing (autoprocessing) of the HIV protease precursor releases the mature, catalytically active HIV protease. Accordingly, the cleaved peptide bond is between the phenylalanine residue at the carboxy terminus and the proline residue at the amino terminus of the mature HIV protease domain. Nucleotide sequences that encode the transframe region and that encode the mature, catalytically active HIV protease (99 amino acids), and the amino acid sequences thereof, are known in the art and may be found in any number of publications (see, e.g., Konvalinka et al., J. Virol. 69:7180-86 (1995) GenBank Accession No. AY352051-AY32073); Winslow et al., AIDS Res. Hum. Retroviruses 11:107-13 (1995); Candotti et al., C.R. Acad. Sci. III, Sci. Vie 317:183-89 (1994); see e.g.; GenBank Accession No. NP_{—}705926; GenBank Accession Nos. U12743 and AAA21287; GenBank Accession No. NP_{—}787043). (See also the entire genome of an HIV isolate, which is provided, for example, in GenBank Accession No. NC_{—}001802.)

The mature HIV-1 protease is an obligatory dimer of identical 11-kdalton subunits that each contribute one of the two catalytic aspartic residues (see, e.g., Paulus et al., J. Biol. Chem. 274:21539-43 (1999)). The transframe region includes the transframe peptide (TFP) (eight arniinoacids) and a 48-amino acid p6^{pol} protein (see, e.g., GenBankAccession No. NP_{—}787043). The nucleotide sequences that encode the HIV protease and the transframe region may be isolated, for example, by amplification using PCR techniques and oligonucleotide primers (see, e.g., Example 2 herein), from any HIV isolate according to standard molecular biology techniques practiced in the art. Other viruses that contain a transframe region that may be encoded by the polynucleotides and recombinant expression constructs described herein include HIV-2 and HTLV. Any one of these transframe domains may be fused to a sequence of aprotease of interest (e.g., a protease from an infectious disease organism).

In certain embodiments, a polynucleotide encodes an HIV protease mutant. As described herein, use of HIV protease inhibitors has caused the rapid emergence of drug-resistant HIV proteases, which contain one or more substitutions, insertions, or deletions of amino acids in the wildtype HIV protease amino acid sequence. The nucleotide sequence for such HIV protease mutants may be determined according to methods described herein and known in the art, and a polynucleotide encoding such a mutant may subsequently be used in the methods and compositions described herein. Accordingly, a polynucleotide encoding an HIV protease includes a polynucleotide variant that encodes an HIV protease variant or mutant. Polynucleotide variants or mutants may contain one or more substitutions, additions, deletions, and/or insertions of nucleotides such that the polynucleotide variant encodes a protease that contains none, or one or more substitutions, additions, deletions, and/or insertions of an amino acid. When compared with a wildtype or known protease, the protease activity of the encoded protease variant or mutant may not be substantially diminished or reduced, or may be partially (fractionally) diminished or reduced, or may be eliminated or abrogated. The effect on the activity of the encoded protease may generally be determined according to the methods described herein and known in the art (see, e.g., Cheng et al., Antimicrob. Agents Chemother. 48:2437-47 (2004) and references cited therein; Brik et al., Chem. Biol. 9:891-96 (2002)).

Polynucleotides encoding HIV protease mutants (variants) preferably exhibit at least about 70%, 75%, 80%, 85%, 88%, 90%, 92%, 95%, 96%, 98%, or 99% identity to a polynucleotide sequence that encodes a native or wildtype HIV protease or a portion thereof. The percent identity may be readily determined by comparing sequences using computer algorithms well known to those having ordinary skill in the art, such as Align or the BLAST algorithm (Altschul, J. Mol. Biol. 219:555-65 (1991); Henikoff et al., Proc. Nat'l Acad. Sci. USA 89:10915-19 (1992)), which is available at the NCBI website. Default parameters may be used. Similarly, the percent identify of the encoded polypeptide (which may be about at least about 70%, 75%, 80%, 85%, 88%, 90%, 92%, 95%, 96%, 98%, or 99% identity to a native or wildtype HIV protease) may be determined by comparing amino acid sequences using. such computer algorithms. In addition, polynucleotide variants are capable of hybridizing under moderately stringent conditions to a naturally occurring DNA or RNA sequence encoding a native or wildtype HIV protease (or a complementary sequence). Suitable moderately stringent conditionsinclude, for example, pre-washing in a solution of 5×SSC, 0.5% SDS, 1.0 mM EDTA (pH 8.0); hybridizing at 50° C.-70° C., 5×SSC for 1-16 hours; followed by washing once or twice at 22° C.-65° C. for 20-40 minutes with one or more each of 2×, 0.5× and 0.2×SSC containing 0.05-0.1% SDS. For additional stringency, conditions may include a wash in 0.1×SSC and 0.1% SDS at 50°-60° C. for 15 minutes. As known to persons having ordinary skill in the art, variations in stringency of hybridization zonditions may be achieved by altering the time, temperature, and/or concentration of the solutions used for pre-hybridization, hybridization, and wash steps. Suitably stringent conditions can be readily selected without undue experimentation when a desired selectivity of the probe is identified.

Persons having ordinary skill in the art will also readily appreciate that, as a result of the degeneracy of the genetic code, many nucleotide sequences may encode the polypeptides described herein. Some of these polynucleotides bear minimal homology to the nucleotide sequence of any native gene or mRNA. Also, polynucleotides that vary due to differences in codon usage are specifically contemplated.

Modification of a polynucleotide may be performed by a variety of methods, including site-specific or site-directed mutagenesis of DNA encoding the polypeptide of interest (e.g., a protease. including an HIV protease or a reporter polyeptide) or by using DNA amplification methods using primers to introduce and amplify alterations in the DNA template, such as PCR splicing by overlap extension (SOE). Site-directed mutagenesis is typically effected using a phage vector that has single- and double-stranded forms, such as M13 phage vectors, which are well-known and commercially available. Other suitable vectors that contain a single-stranded phage origin of replication may be used (see, e.g., Veira et al., Meth. Enzymol. 15:3, 1987). In general, site-directed mutagenesis is performed by preparing a single-stranded vector that encodes the polypeptide of interest. An oligonucleotide primer that contains the desired mutation within a region of homology to the DNA in the single-stranded vector is annealed to the vector followed by addition of a DNA polymerase, such as E. coli DNA polymerase I (Klenow fragment), which uses the double stranded region as a primer to produce a heteroduplex in which one strand encodes the altered sequence and the other the original sequence. Additional disclosure relating to site-directed mutagenesis may be found, for example, in Kunkel et al. (Methods in Enzymol. 154:367 (1987)); Kramer et al. (Nucleic Acids Res. 12:9441 (1984); Kunkel (Proc. Natl Acad. Sci. USA 82:488-92 (1985)), and in U.S. Pat. Nos. 4,518,584 and 4,737,462. The. heteroduplex is introduced into appropriate bacterial cells, and clones that include the desired mutation are selected. The resulting altered DNA molecules may be expressed recombinantly in appropriate host cells to producethe modified protein. Other methods known in the art for introducing mutations (insertions, substitutions, deletions) include oligonucleotide-directed mutagenesis, linker-scanning mutagenesis, mutagenesis using the polymerase chain reaction, and the like (see Ausubel (1995) at pages 8-10 to 8-22; and McPherson (ed.), Directed Mutagenesis: A Practical Approach (IRL Press 1991)).

The nucleotide sequence (derived from the lacZ gene) that encodes β-galactosidase is well known in the art (see Fowler and Zabin, J Biol: Chem. 253:5521-25 (1978)). As described herein and known in the art, the enzyme is encoded by the lacZ gene of the lac operon (see Kalnins et al., EMBO J. 2:593-97 (1983); see, e.g., GenBarik Accession No. V00296). The sequence is commonly incorporated into expression vectors that may be obtained from commercial vendors (for example, pBAD TOPO® LacZ, Invitrogen Life Technologies, Carlsbad, Calif.). As described herein, a nucleotide sequence that encodes a protease recognition sequence is inserted into the nucleotide sequence encoding β-galactosidase by using methods described herein and routinely practiced by persons having skill in the art.

For a given protease, any recognition sequence of the prptease may be used to construct a recombinant reporter polypeptide. The protease recognition sequence may be a portion of a naturally occurring protease substrate or may be an artificial polypeptide. For instance, the cleavage sites of HIV protease including the following peptides,which may be included in a protease recognition sequence:

(SEQ ID NO:11)

| | |
|---|---|
| P17/p24: | Ser Gln Asn Tyr Pro Ile Val Gln |

(SEQ ID NO:12)

| | |
|---|---|
| P24/X: | Ala Arg Val Leu Ala Glu Ala Met |

(SEQ ID NO:13)

| | |
|---|---|
| X/p7 | Ala Thr Ile Met Met Gln Arg Gly |

(SEQ ID NO:14)

| | |
|---|---|
| P7/p6: | Pro Gly Asn Phe Leu Gln Ser Arg |

(SEQ ID NO:15)

| | |
|---|---|
| P6/PR: | Ser Phe Asn Phe Pro Gln Ile Thr |

(SEQ ID NO:16)

| | |
|---|---|
| PR/RT: | Thr Leu Asn Phe Pro Ile Ser Pro |

(SEQ ID NO:17)

| | |
|---|---|
| RT5/RNase H: | Ala Glu Thr Phe Tyr Val Asp Gly |

(SEQ ID NO:18)

| | |
|---|---|
| RT/IN: | Arg Lys Ile Leu Phe Leu Asp Gly |

(SEQ ID NO:19)

| | |
|---|---|
| DEG1: | Gln Ile Thr Leu Trp Gln Arg Pro |

(SEQ ID NO:20)

| | |
|---|---|
| DEG2: | Asp Thr Val Leu Glu Glu Met Ser |

(SEQ ID NO:21)

| | |
|---|---|
| DEG3: | Asp Gln Ile Leu Ile Glu Ile Cys |

In one embodiment the nucleotide sequence encodes an HIV protease recognition sequence, which comprises the amino acid sequence VSFNFPQITL (SEQ ID NO: 1). This HIV protease recognition sequence spans the p6/PR junction of the HIV gag/pol polyprotein that is efficiently cleaved by the HIV protease (see, e.g., Krausslich et al., Proc. Nat'lAcad. Sci. USA 86:807-11 (1989); Baum et al., Proc. Nat'l Acad. Sci. USA 87:10023-27 (1990)). In certain embodiments, the HIV protease recognition sequence (which may include any one of SEQ ID NOS:11-21) may comprise less than ten amino acids, and may comprise nine, eight, or seven amino acids (see also, e.g., Billich et al., J. Biol. Chem. 263:17905-908 (1988); Moore et al., Biochem. Biophys. Res. Commun. 159:420-25 (1989); Darke et al., Biochem. Biophys. Res. Commun. 156:297-303 (1988); and Krausslichet al., supra).

A protease recognition sequence, such as an HIV protease recognition sequence is inserted into a reporter polypeptide at a position such that the reporter activity remains functional and detectable by methods for determining the reporter activity. When the reporter polypeptide is cleaved at or near the recognition sequence, the reporter activity is significantly diminished or reduced or may be totally eliminated. By way of example, a protease recognition sequence may be inserted into the Saul site of the β-glactosidase-encoding nucleotide sequence of E. coli (see Baum et al., supra; Cheng et al., Antimicrob. Agents Chemother. 48:2437-47.(2004); Example 2). As described herein and described in the art, the encoded β-glactosidase reporter polypeptide that includes the HIV protease recognition sequence inserted at the SauI site retains β-galactosidase reporter activity. Cleavage of the thus modified β-galactosidase by HIV protease that is co-expressed in E. coli causes the loss of β-galactosidase activity (see, e.g., U.S. Pat. No. 5,436,131; EP Pat. Appl. No. 0 421 109; and Baum et al., supra).

Nucleotide sequences as described herein may be joined to a variety of other nucleotide sequences using established -recombinant DNA techniques. For example, a polyhucleotide may be cloned into any of a variety of cloning vectors, including plasmids, phagemids, phage derivatives (e.g., lambda phage derivatives), and cosmids. Vectors of particular interest include expression vectors, replication vectors, probe generation vectors, and sequencing vectors. In general, a suitable vector contains an origin of replication functional in at least one organism, convenient restriction endonuclease sites, and one or more selectable markers, and suitable regulatory sequences. Other elements will depend upon the desired use, and will be apparent to those having ordinary skill in the art.

The polynucleotides described herein may be inserted into a recombinant expression vector (construct). The polynucleotide that encodes the precursor of the protease and the polynucleotide that encodes the reporter polypeptide comprising the protease recognition sequence may be incorporated into a single recombinant expression vector or into two distinct expression vectors, which are co-transfected or co-transformed into a host cell. When the nucleotide sequence encoding the protease precursor and the nucleotide sequence encoding the reporter polypeptide are present in the same polynucleotide, the nucleotide sequences encoding these distinct polypeptides may be operatively linked to a single promoter. Alternatively, the nucleotide sequence encoding the protease precursor may be operatively linked to a first promoter, and the nucleotide sequence encoding the reporter polypeptide may be operatively linked to a second promoter. The first and second promoters may be the same or may be different promoters. In a particular embodiment, the nucleotide sequence encoding the protease precursor and the nucleotide sequence encoding the reporter polypeptide are operatively linked to, and thus regulated by, a single promoter, which permits coordination of expression levels of both polypeptides. When the nucleotide sequence encoding the protease precursor and the nucleotide sequence encoding the reporter polypeptide are operatively linked to different promoters the expression levels of the precursor protease polypeptide and the reporter polypeptide are differentially regulated.

When the nucleotide sequences are under the control of a single promoter and the host cells are prokaryotic cells, the nucleotide sequences may be arranged in the polynucleotide such that when the sequences are transcribed, a polycistronic mRNA is formed. The polycistronic mRNA, in this instance, a two-cistron mRNA, is in turn translated to provide the protease precursor and the reporter polypeptide. Alternatively, the nucleotide sequences may be arranged into a nucleotide expression cassette so that the cassette encodes a polyprotein that comprises the reporter polypeptide with a protease recognition sequence, and encodes the protease precursor of interest, wherein the engineered reporter polypeptide and the protease precursor are linked with each other via a recognition sequence of the protease. The resulting polyprotein may be autoproteolytically processed (self-processed or auto-processed) into individual protein components, or fragments thereof, by the action of its protease portion at or near the protease recognition sequences within the junction between the individual protein components and within the reporter polypeptide. The above nucleotide expression cassette is also applicable to screening systems that use a eukaryotic cell as host cell.

In general, recombinant expression vectors invention will also contain one or more nucleotide sequences comprising regions (suitable regulation control sequences) necessary for transcription and translation, such as a promoter, enhancer, transcription initiation site, transcription termination site, translation initiation site (ribosome binding site), etc. (see, e.g., Goeddel, Gene Expression Technology in 185 METHODS IN ENZYMOLOGY (1990)). The choice of promoter will depend upon the cell type to be transformed and the degree or type of control desired. Promoters can be constitutive or active and may further be cell type specific, tissue specific, individual cell specific, event specific, temporally specific, or inducible.

Examples of constitutive or nonspecific promoters included in recombinant expression vectors for transfection into eukaryotic cells include the SV40 early promoter (U.S. Pat. No. 5,118,627), the SV40 late promoter (U.S. Pat. No. 5,118,627), CMV early gene promoter (U.S. Pat. No. 5,168,062), and adenovirus promoter. In addition to viral promoters, cellular promoters may be used. In particular, cellular promoters for the so-called housekeeping genes are useful. Viral promoters are preferred, because generally they are stronger promoters than cellular promoters. Promoter regions have been identified in the genes of many eukaryotes including higher eukaryotes, such that suitable promoters for use in a particular host can be readily selected by those skilled in the art. Inducible promoters may also be used. These promoters include MMTV LTR (PCT Publication No. WO 91/13160), inducible by dexamethasone; metallothionein promoter, inducible by heavy metals; and promoters with cAMP response elements, inducible by cAMP. By using an inducible promoter, which is a regulated promoter, the nucleic acid sequence encoding the polypeptides described herein maybe delivered to a cell by the recombinant expression construct and will remain quiescent until the addition of the inducer. This allows further control on the timing of production of the gene product.

Representative examples of promoters used in bacterial expression systems include the E. coli araBAD (inducible by arabinose), lac or trp, Tac, the phage lambda P_{L} promoter, and other promoters known to control expression of polynucleotides in prokaryotic cells. Other bacterial promoters include lacI, lacZ, T3, T5, T7, gpt, and lambda P_{R}. An expression vector may also include a combination of promoters such as T7 and lac. Selection of the appropriate vector and promoter is well within the level of ordinary skill in the art. The promoter may be a regulated promoter, which may be tightly regulated, that is, the promoter is specifically inducible and permits little or no transcription of nucleotide sequences under its control in the absence of an induction signal, as is known to those familiar with the art (see, e.g., Guzman et al., J. Bacteriol. 177:4121 (1995)); Carra et al., EMBO J. 12:35 (1993); Mayer, Gene 163:41 (1995); Haldimann et al. J. Bacteriol. 180:1277(1998); Lutz et al., Nucleic Acids Res. 25:1203 (1997); Allgood et al., Curr. Opin. Biotechnol. 8:474 (1997); and Makrides, Microbiol. Rev. 60:512 (1996)). A regulated promoter may be a promoter as provided herein and may also be a repressor binding site, an activator binding site, or any other regulatory sequence that controls expression of a nucleotide sequence as provided herein.

Host cells are genetically engineered (transduced, transformed, or transfected) with the vectors and/or recombinant expression constructs described herein, which may be, for example, a cloning vector, a shuttle vector, or an expression construct. The recombinant expression vector can be introduced into prokaryotic or eukaryotic cells via conventional transformation or transfection techniques. As used herein, the terms transformation and transfection are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al., supra, and other laboratory manuals.

The vector or construct may be, for example, in the form of a plasmid, cosmid, a viral particle, a phage, etc. The engineered host cells can be cultured in conventional nutrient media modified as appropriate for activating or inducing promoters, selecting transformants, or amplifying particular nucleotide sequences such as those encoding a protease precursor and a reporter polypeptide as described herein. The culture conditions for particular host cells selected for expression, such as temperature, pH, media, and the like, will be readily apparent to the ordinarily skilled artisan. Suitable prokaryotic hosts for transformation or transfection include E. coli, Bacillus subtilis, S. typhimurium and various species within the genera Pseudomonas, Streptomyces, and Staphyloicoccus, although others may also be employed as a matter of choice. Any other plasmid or vector may be used as long as they are replicable and viable in the host. Other host cells include fungal cells, such as yeast; insect cells, such as Drosophila S2 and Spodoptera SJ9; animal cells, such as CHO, COS, or 293 cells; plant cells, or any suitable cell already adapted to in vitro propagation. The selection of an appropriate host is deemed to be within the scope of those skilled in the art from the teachings herein.

Methods and Agents

In certain embodiments, a method is provided for identifying an agent that alters (increases or decreases in a statistically significant manner, preferably decreases) the proteolytic activity of a protease. In one embodiment, the method described herein may be used to identify an agent or compound that inhibits proteolytic activity of a protease of interest (e.g., an HIV protease). The method for identifying such an agent comprises contacting a candidate agent (for example, a test compound that is a small molecule) and a cell (that is, combining, mixing, adding together, or otherwise permitting interaction between the test compound and the cell). In certain embodiments, the host cell is a bacterial cell and in a particular embodiment, the bacterial host cell is an E. coli cell. The cell is a host cell as described herein that expresses a precursor of a protease and that expresses a reporter polypeptide that comprises a protease recognition sequence, which sequence, when recognized by the protease renders the reporter polypeptide susceptible to cleavage (proteolysis) by the protease. In one embodiment, the protease precursor is autoproteolytically cleaved to release the catalytically active protease, which then can hydrolyze the reporter polypeptide at or near the protease recognition sequence. The reporter activity of the reporter polypeptide can then be detected and compared in the absence and presence of the candidate agent to determine the capability of the agent to inhibit the proteolytic activity of the protease of interest. Measuring the reporter activity of the reporter polypeptide (and thus determining the level of protease activity) may be carried out, for example either continuously or at a fixed time point after combining the host cell and the candidate agent. In certain embodiments, expression of the protease precursor and the reporter polypeptide is under the control of an inducible promoter. The compound capable of inducing expression may be added to the mixture of the host cell and candidate agent concurrently, prior to, or after addition of the agent (or compound known to inhibit the proteolytic activity of the protease).

In a certain embodiment, a method is provided for identifying an agent that inhibits catalytic activity of an HIV protease. The steps of such a method include contacting a candidate agent and a cell (that is, combining, mixing, adding together, or otherwise permitting interaction between the test compound and the cell). The cell may be a bacterial cell, which may be an E. coli cell, or a S. typhimurium cell, or a B. subtilis cell, that expresses a precursor of an HIV protease and that expresses a reporter polypeptide, such as β-galactosidase, that contains an HIV protease recognition sequence (e.g., VSFNFPQITL (SEQ ID NO: 1). As described herein, the cell may be transfected or transformed with a recombinant expression vector comprising at least one promoter that is operatively linked to a nucleotide sequence that encodes the precursor of an HIV protease wherein the precursor comprises an amino acid sequence that includes a transframe region and the mature HIV protease. In certain embodiments, the same recombinant expression vector also comprises a nucleotide sequence that encodes the reporter polypeptide that has inserted into its sequence an HIV protease recognition sequence and that may be operatively linked to the same at least one promoter that is operatively linked to the HIV protease precursor, or may be operatively linked to a second promoter (which could be a different or the same type of promoter operatively linked to the HIV protease precursor). The step of contacting occurs under conditions and for a time sufficient to permit expression and autoproteolytic processing of the HIV precursor polypeptide to yield the mature, catalytically active HIV protease, and that permits cleavage (proteolysis) of the reporter polypeptide at or near the HIV protease recognition site that is within the reporter polypeptide.

As described herein, the candidate agent may be contacted with the cell, prior to, at the same time, or after the cell is induced to express the polypeptide(s) encoded by the recombinant expression vector (i.e., addition of a compound that is an inducing agent appropriate for a particular promoter such as arabinose to induce the pBAD promoter or IPTG to induce the lac promoter). The reporter activity is then detected according to methods known in the art and described herein. When the reporter polypeptide is β-galactosidase, the reporter activity is the catalytic activity of the β-galactosidase enzyme. Accordingly, added to the cells is a β-galactosidase substrate, that is a β-D-galactopryanoside or a synthetic analog thereof, such as a synthetic analog of lactose (including chromogenic substrates such as ONPG and X-gal and fluorogenic substrates such as MUG and DifMuG. In certain embodiments the bacterial cell is made permeable for the entry of the β-galactosidase substrate, which may be accomplished by adding one or more compounds and detergents known to permeabilize a bacterial membrane, including but not limited to polymyxin B sulfate and Triton X-100. The reporter activity that is determined in the presence of the candidate agent and that is determined in the absence of the candidate agent may then be compared. An increase in the reporter activity (which corresponds to a decrease in the proteolysis of the reporter polypeptide) in the presence of the candidate agent compared with the activity in the absence of the agent indicates that the agent is an inhibitor of the protease.

The amount of a known inhibitor of a protease (such as an HIV protease) or a candidate agent that may be capable of inhibiting protease activity of the protease of interest may range from about at least 0.01 nM to about 5000 μM. A bioactive agent may include, for example, a peptide, a polypeptide (for example, a ligand that binds to a protease, such as the HIV protease), an oligonucleotide or polynucleotide, antibody or binding fragment thereof, lipid, hormone, or small molecule. Candidate agents for use in a method of screening for a bioactive agent that is capable of altering (increasing or decreasing in a statistically significant manner) protease activity include an inhibitor of HIV protease activity. Inhibitors may inhibit, slow, hinder, impair, suppress, or otherwise decrease (e.g., in a statistically significant manner) the autoproteolytic and/or proteolytic of a protease of interest, including an HIV protease. The candidate agents may be provided as “libraries” or collections of compounds, compositions, or molecules. Such molecules typically include compounds known in the art as “small molecules” that have molecular masses less than 10⁵ daltons, less than 10⁴ daltons, or less than 10³ daltons. Candidate agents further may be provided as members of a combinatorial library, which includes synthetic agents prepared according to a plurality of predetermined chemical reactions performed in a plurality of reaction vessels. For example, various starting compounds may be prepared employing one or more of solid-phase synthesis, recorded random mix methodologies and recorded reaction split techniques that permit a given constituent to traceably undergo a plurality of permutations and/or combinations of reaction conditions. The resulting products comprise a library that can be screened and then followed by iterative selection and synthesis procedures to provide, for example, a synthetic combinatorial library of peptides (see, e.g., PCT/US91/08694, PCT/US91/04666) or other compositions that may include small molecules as provided herein (see, e.g., PCT/US94/08542, U.S. Pat. No. 5,798,035, U.S. Pat. No. 5,789,172, U.S. Pat. No. 5,751,629). Those having ordinary skill in the art will appreciate that a diverse assortment of such libraries may be prepared by a skilled artisan according to established procedures (see, e.g., Example 5 herein).

Bioactive agents that are believed to or are known to interact with a protease, such as the HIV protease, may be included in the methods described herein to identify an agent that inhibits the protease. Known HIV protease inhibitors include those that are currently used for treating patients with HIV infection or AIDS, and include amprenavir (APV) (Agenerase®, GlaxoSmithKline, Research Triangle Park, N.C.); fosamprenavir (Lexiva®, GlaxoSmithKline); indinavir (IDV) (Crixivan®, Merck Pharmaceuticals, Whitehouse Station, N.J.); nelfinavir (NFV) (Viracept®, (Agouron Pharmaceuticals) Pfizer Inc., New York, N.Y.); ritonavir (RTV) (Norvir®, Abbott Laboratories, Abbott Park, Ill.); and saquinavir (SQV) (Fortovase®, Hoffman-La Roche, Basel, Switzerland). Another protease inhibitor that inhibits HIV protease is pepstatin A, which is a peptide-based aspartic protease inhibitor. In the methods for identifying an agent that inhibits catalytic activity of HIV protease as described herein, pepstatin A may serve as a control for cell permeability. In one embodiment, Pepstatin A is contacted with a cell that is a bacterial cell such as an E. coli cell. Pepstatin A is unable to diffuse into or in some manner be transported across the bacterial cell membrane and therefore cannot, within the cell, inhibit autoproteolytic processing of the HIV protease precursor and/or inhibit proteolytic cleavage of the reporter polypeptide that comprises an HIV protease recognition sequence. Thus, lack of inhibition of HIV protease activity by pepstatin A demonstrates that the integrity of the bacterial cell has not been compromised or adversely affected.

The methods described herein are useful for determining a drug resistance profile of a particular viral isolate, such as an HIV isolate. In one embodiment, the methods may be used to identify HIV protease mutants and/or to determine the level of sensitivity of these mutants to known HIV protease inhibitors such as APV, fosamprenavir, IDV, NFV, RTV, and SQV or any other HIV protease inhibitor that is currently used to treat subjects infected with HIV or is currently being tested in preclinical or clinical trials for treating HIV infection. Thus, the methods described herein may be used for determining the sensitivity (or the resistance) of an HIV strain or isolate obtained from a subject who is infected with HIV. Determining the sensitivity of these mutants and isolates to particular HIV protease inhibitors, in other words determining the level of drug resistance of these mutants, is useful to a person skilled in the medical art so that the skilled person may provide the a therapeutic regimen that is most effective for the subject who is infected with HIV. In still another embodiment, the method may be used to determine the level of sensitivity of a particular HIV strain that is isolated from a subject to any one of a number of different protease inhibitors that are available for treating patients infected with HIV, who may or may not have yet developed AIDS. The caregiver can then determine an appropriate therapeutic regimen for the patient.

Accordingly, in one embodiment, a method is provided for determining sensitivity of a human immunodeficiency virus (HIV) isolate to an inhibitor of HIV protease activity comprising the steps of preparing a host cell that comprises a recombinant expression vector comprising a promoter operatively linked to (i) a nucleotide sequence that encodes a precursor of the HIV protease, which protease is from the HIV isolate (i.e., an HIV transframe region fused to the nucleotide sequence encoding the HIV protease from the HIV isolate) and (ii) a nucleotide sequence that encodes a reporter polypeptide (such as β-galactosidase) comprising a protease recognition sequence (for example, an HIV protease recognition sequence as described herein). As described herein, a host cell may be prepared according to methods described herein and practiced in the art, for example, by transforming or transfecting the host cell (for example, a bacterial cell, such as an E. coli cell) with a recombinant expression construct using methods commonly and routinely practiced by persons skilled in the molecular biology art.

The method further comprises contacting an inhibitor of HIV protease activity with the host cell (that is, combining, mixing, adding together, or otherwise permitting interaction between the test compound and the cell), under conditions and for a time that permit expression and autoproteolytic processing of the precursor of the HIV protease and cleavage of the reporter polypeptide by the HIV protease and then detecting a reporter activity of the reporter polypeptide. The level of reporter activity of the reporter polypeptide in the presence of the inhibitor is compared with the level of reporter activity in absence of the inhibitor of HIV protease activity, wherein an alteration in the level of activity (increase or decrease that is statistically significant) of the reporter polypeptide in the presence of the inhibitor of HIV protease activity compared with the level of activity of the reporter polypeptide in the absence of the inhibitor of HIV protease activity indicates the sensitivity of the HIV protease from the HIV isolate to the inhibitor of HIV protease activity. The degree (or level or extent) of sensitivity or resistance of the HIV protease from the HIV isolate may also be compared with the sensitivity of a known HIV isolate to the HIV inhibitor. The methods described herein, particularly in a high-throughput format, may be used for screening an HIV isolate or multiple isolates against at least one, two, three, four, five, six, or more HIV protease inhibitors.

In one embodiment, the HIV isolate may be obtained or purified from a biological sample, and the nucleotide sequence encoding the HIV protease from the particular HIV isolate may be determined according to methods described herein and practiced in the art. The HIV isolate may be obtained from a biological sample, which as used herein refers to a sample containing at least one HIV isolate, HIV polypeptide, andor HIV polynucleotide, and may be provided by obtaining a blood sample, biopsy specimen, tissue explant, organculture, or any other tissue or cell preparation from a subject or a biological source. A sample may further refer to a tissue or cell preparation in which the morphological integrity or physical state has been disrupted, for example, by dissection, dissociation, solubilization, fractionation, homogenization, biochemical or chemical extraction, pulverization, lyophilization, sonication, or any other means for processing a sample derived from a subject or biological source. The subject or biological source may be a human or non-human animal, a primary cell culture, or culture adapted cell line. In particular embodiments, the biological sample is obtained from a subject (for example, a subject who is infected with HIV) and includes but is not limited to blood, serum, saliva, semen, vaginal secretions, pulmonary fluid or wash, ascites, and a nasopharyngeal wash. Methods for isolating or separating a virus such as HIV from a biological sample under appropriate containment and safety protocols are routinely practiced by persons skilled in the art.

Assay System

In another embodiment an assay system is provided that is useful for identifying an agent that inhibits a protease. Such an assay system comprises a cell that expresses a precursor of a protease and a recombinant reporter polypeptide that contains a protease recognition sequence, a compound that induces expression, and a substrate of the reporter polypeptide, all of which are described in detail herein. The protease may be a protease from an infectious disease organism such as a virus (e.g., HIV-1, HIV-2, HTLV, poliovirus) or a bacterium. The precursor protease may be comprised of a precursor domain or region that is fused to the active protease domain. An example of a precursor domain is the transframe region of a virus, such as the transframe region of HIV or of HTLV. In certain other embodiments the assay system comprises an agent that inhibits the protease activity or that is being tested for its capability to inhibit the protease.

In a particular embodiment, the assay system comprises a cell that expresses an HIV protease precursor and a recombinant reporter polypeptide that contains a protease recognition sequence, a compound that induces expression, and a substrate of the reporter polypeptide, and in certain embodiments an agent that inhibits HIV protease activity, all of which are described in detail herein. Accordingly, the assay system comprises (a) a cell comprising a recombinant expression vector as described herein, which includes a promoter operatively linked to (i) a nucleotide sequence that encodes an HIV transframe region fused to the nucleotide sequence of an HIV protease to provide a precursor of the HIV protease and (ii) a nucleotide sequence that encodes a reporter polypeptide that comprises a protease recognition sequence; (b) a compound that induces expression of the precursor of the HIV protease and the reporter polypeptide; and (c) a reporter polypeptide substrate.

As described herein the cell may be a bacterial cell, which may be an E. coli cell. The recombinant expression construct may be a 2-cistron construct, wherein a single promoter is operatively linked to the nucleotide sequence that encodes the HIV protease precursor and to the nucleotide sequence that encodes the reporter polypeptide that contains the protease recognition sequence. In an alternative embodiment, one promoter is operatively linked to the nucleotide sequence that encodes the HIV protease precursor and a second promoter is operatively linked to the nucleotide sequence that encodes the reporter polypeptide. The first and second promoters may be the same promoter or may be different promoters. The promoters may be one of several suitable bacterial promoters described herein and known in the art. In certain embodiments the reporter polypeptide is β-galactosidase and the protease recognition sequence is an HIV protease recognition sequence such as VSFNFPQITL (SEQ ID NO: 1). As described herein the HIV protease recognition sequence may comprise less than ten amino acids, and may comprise nine, eight, or seven amino acids (see, e.g., Billich et al., J. Biol. Chem. 263:17905-908 (1988); Moore et al., Biochem. Biophys. Res. Commun. 159:420-25 (1989); Darke et al., Biochem. Biophys. Res. Commun. 156:297-303 (1988); and Krausslich et al., supra). The promoter(s) may be inducible promoters, and accordingly a compound that induces expression of the polynucleotides that encode the HIV precursor polypeptide and the reporter polypeptide, that is, a compound induces transcription of nucleotides and thus controls polypeptide expression are discussed herein. Substrates of β-galactosidase include ONPG, DifMuG, and MUG.

The assay system may further comprise an inhibitor of an HIV protease, such as an inhibitor that is used for treating a patient who is infected with HIV or who has AIDS, such as those described herein and known in the medical art. Alternatively, the assay system may comprise an agent that is being screened for its capability to inhibit an HIV protease. In another embodiment, the assay system comprises a library of agents or test compounds for screening to identify an agent that inhibits HIV protease activity. Exemplary types of bioactive agents and compound libraries are described in detail herein.

This assay system and the methods described herein will have significant value in high-throughput screening, that is, in automated screening of a large number of candidate compounds for activity against one or more proteases, such as HIV proteases. The assay system and methods have particular value, for example, in screening synthetic or natural product libraries for compounds that exhibit inhibitory activity that affects proteolytic activity of a protease of interest. The assay system and methods described herein are therefore amenable to automated, cost-effective, high-throughput drug screening and have immediate application in a broad range of pharmaceutical drug development programs. In one embodiment, the compounds to be screened are organized in a high-throughput screening format such as a 96-well plate format, or other regular two-dimensional array, such as a 384-well, 48-well or 24-well plate format or an array of test tubes. In certain embodiments of such analytical methods, particularly a high-throughput method, an automated apparatus is useful and is under the control of a computer or other programmable controller. The controller can continuously monitor the results of each step of the process and can automatically alter the testing paradigm in response to those results. Sensitivity and detection limits may vary as a function of the reporter activity and reporter signal that is monitored and further with regard to assay formats, such as conventional test tubes or high-throughput formats such as 96-well, 384-well or other high-throughput microplates, or other container vessels designed for high-throughput assay formats.

High-throughput assays (or multiplexing) allow testing of multiple HIV isolates and/or multiple HIV protease inhibitors and/or candidate agents simultaneously using the methods described herein. In certain embodiments, multiple expression vectors may be constructed wherein each vector (recombinant expression construct) comprises a polynucleotide encoding an HIV protease from a different HIV isolate, which may include HIV protease mutants or variants. Each expression vector may be transfected or transformed into a host cell, such as a bacterial cell (for example, E. coli), providing multiple (two or more) host cells for use in the methods described herein. Thus, a single agent or multiple candidate agents may then be screened for their capability to inhibit HIV proteases prepared from multiple HIV isolates. In one embodiment, a method is provided for the multiplex identification of an agent that inhibits an HIV protease comprising contacting (that is, combining, mixing, adding together, or otherwise permitting interaction) each candidate agent with each host cell with a cell that expresses a precursor of an HIV protease and that expresses a reporter polypeptide, wherein the reporter polypeptide comprises an HIV protease recognition sequence, under conditions and for a time that permit expression and autoproteolytic processing of the precursor of the HIV protease and cleavage of the reporter polypeptide by the HIV protease; detecting the reporter activity of the reporter polypeptide; and comparing the reporter activity of the reporter polypeptide in the presence and absence of the candidate agent; wherein an increase in activity of the reporter polypeptide in the presence of the candidate agent compared with activity of the reporter polypeptide in the absence of the candidate agent indicates that the candidate agent inhibits the HIV protease. In certain embodiments, the reporter polypeptide is β-galactosidase. In particular embodiments, the HIV protease recognition sequences comprises the amino acid sequence set forth in SEQ ID NO:1 or may comprise an HIV protease recognition sequence set forth in any one of SEQ ID NOS:11-21.

The methods described herein may also be used for multiplex identification of the sensitivity (or the resistance) of an HIV isolate to two or more inhibitors of HIV protease activity. Such a method for multiplex determination of sensitivity of an HIV isolate to two or more inhibitors of HIV protease activity comprises (a) preparing a host cell that comprises a recombinant expression vector comprising a promoter operatively linked to (i) a nucleotide sequence that encodes an HIV transframe region fused to the nucleotide sequence encoding the HIV protease from the HIV isolate and (ii) a nucleotide sequence that encodes a reporter polypeptide comprising a protease recognition sequence; (b) contacting each inhibitor of HIV protease activity with the host cell (that is, combining, mixing, adding together, or otherwise permitting interaction between the inhibitor (or test compound or agent) and the cell), under conditions and for a time that permit expression and autoproteolytic processing of the precursor of the HIV protease and cleavage of the reporter polypeptide by the HIV protease; (c) detecting a reporter activity of the reporter polypeptide; and (d) comparing the level of reporter activity of the reporter polypeptide in the presence and absence of the inhibitor of HIV protease activity, wherein an alteration in the level of reporter activity of the reporter polypeptide in the presence of the inhibitor of HIV protease activity compared with level of reporter activity of the reporter polypeptide in the absence of the inhibitor of HIV protease activity indicates the level of sensitivity of the HIV protease from the HIV isolate to the inhibitor of HIV protease activity. In certain embodiments, the reporter polypeptide is galactosidase. In particular embodiments, the HIV protease recognition sequences comprises the amino acid sequence set forth in SEQ ID NO:1 or may comprise an HIV protease recognition sequence set forth in any one of SEQ ID NOS:11-21.

The components of the assay system may be provided separately or may be provided together, such as in a kit. Components of the assay system may be prepared and included in a kit according to methods that maximize the stability of the individual components, which methods persons skilled in the art are familiar, such that the assay system yields reproducible and accurate data. For example, cells of the assay system that are bacterial host cells, may be provided as a suspension, lyophilized, or as an agar stab. Additional components of the system may also be included such as buffers, containers for mixing the assay components such as microplates or test tubes, and instructions for performing the assay.

The following Examples are offered by way of illustration and not by way of limitation.

### EXAMPLE 1

### Materials and Reagents

The following materials and reagents were used in the Examples described herein.

The bacterial expression vector pBAD-TOPO-LacZ was obtained from Invitrogen Corp. (Carlsbad, Calif.). HIV-1I NL4.3 DNA was a gift from Jiing-Kuan Yee (The City of Hope, Duarte, Calif.). All DNA-modifying enzymes were from New. England Biolabs (Beverly, Mass.), except for Taq polymerase, which was from Panvera (Madison, Wis.). Oligonucleotides for PCRs were synthesized by MWG Biotech (High Point, N.C.), and the amplified DNA fragments were then purified with ZymoClean (Zymo Research, Orange, Calif.). Pepstatin A was obtained from ICN Biochemicals (San Diego, Calif.). FDA-approved HIV protease inhibitors, including amprenavir (APV); indinavir (IDV); helfinavir (NFV); ritonavir (RTV); and saquinavir (SQV), were obtained through the National Institutes of Health (NIH) AIDS Research and Reference Reagent Program, Divisions of AIDS, National Institute of Allergy and Infectious Diseases, NIH. Analytical thin-layer chromatography was performed on precoatedplates (silica gel 60F-254; Merck & Co., Inc., Whitehouse Station, N.J.). Silica gel used for flash column chromatography was Mallinckrodt type 60 (230 to 400 mesh). Novex Bis-Tris polyacrylamide gels for protein separation were purchased from Invitrogen Corp., and the polyvinylidene fluoride membranes (PVDF) were from Millipore (Bedford, Mass.). Anti-β-galactosidase monoclonal antibodies were purchased from Roche Applied Science (Indianapolis, Ind.), and HIV type 1 (HIV-1) protease antiserum was provided by D. Bailey and Mark Page (Pfizer Inc., New York, N.Y.) through the NIH AIDS Research and Reference Reagent Program, Divisions of AIDS, National Institute of Allergy and Infectious Diseases, NIH. The horseradish peroxidase-conjugated secondary antibodies as well as the Lumi-Glo chemiluminescence reagent were purchased from KPL Inc. (Gaithersburg, Md.). Reagents for the highest purity were purchased from Sigma Aldrich (St. Louis, Mo.); Acros Organics (Belgium); Calbiochem/Novabiochem International (San Diego, Calif.); or Bacflem, Inc. (multiple locations).

### EXAMPLE 2

### Construction of Expression Plasmids

In this Example, construction of recombinant expression vectors for co-expression of the HIV protease and recombinantly engineered β-galactosidase is described.

The pBAD recombinant expression system, which incorporates the BAD promoter for recombinant protein expression, was used to construct all expression plasmnids in this study. To engineer β-galactosidase as a substrate for the HIV protease, a pair of cleavage cassettes, encoding the decapeptide corresponding to the p6/PR cleavage site (Val-Ser-Phe-Asn-Phe-Pro-Gln-Ile-Thr-Leu, SEQ ID NO: 1) of the HIV Gag-Pol polyprotein, were synthesized and ligated with Saup-digested PBAD-TOPO LacZ (Baum et al. supra), thereby inserting the cleavage site at the Saul site (which corresponds to amino acid 131 in this β-galactosidase sequence). The construct was designated pβ-Gal_{PR}.

Co-expression of the HIV protease and β-Gal_{PR} was carried out using a two-cistron approach. The vector is illustrated in FIG. 1A. The first cistron encodes the HIV protease or TF-PR. Before the stop codon of the first cistron was a Shine- Dalgamo sequence of AGGAGGAA for ribosome binding, which was followed with a start codon for translation of the second cistron, β-Gal_{PR}. The coding sequence for the HIV protease gene was amplified by PCR from HIV-1 NL4.3 DNA with oligonucleotide PR-F (ATACCATGGCCCCTCAGATCACTCTTCGGCAGCGACC, SEQ ID NO: 2) as the forward primer and oligonucleotide PR-R (AGCCCATGGGfTATTCCTCCTTAAAATTTA, SEQ ID NO: 3) as the reverse primer. The italicized and underlined nucleotides represent NcoI restriction sites. The coding sequence for the HIV protease precursor, which includes the TF and the protease domain was amplified with oligonucleotide TF-F (CATACCATGGGCTTTTTTAGGGAAGATCTGGCCTTC, SEQ ID NO: 4) as the forward primer and oligonucleotide PR-R as the reverse primer. After digestion with NcoI, the amplified fragments Were then cloned into the unique NcoI site of pβ-Gal^{PR}. The resulting plasmid was designated pPR^{WT} or pTF-PR^{WT}, respectively. E. coli DH5α was used as the host for plasmid preparations and for recombinant protein expression (Sambrook et al., Molecular nd Cloning: A Laboratory Manual (2 ed. 1989).

### EXAMPLE 3

### In Vitro PCR-Mediated Mutagenesis

In this Example, preparation of an HIV protease mutant is described.

A modified form of the HIV protease precursor that contained a D25N mutation at the catalytic residue Asp25 to abolish the proteolytic activity (Kohl et al., Proc. Nat'l Acad. Sci. USA 85:4686-90 (1988)) was constructed by PCR-mediated mutagenesis and was usedas a control (pTF-PR^{D25N})(FIG. 1A) (Ansaldi et al., Anal. Biochem. 234:110-11 (1996)). The PCR-mediated mutagenesis includes three PCRs. The first PCR used pTF-PR^{WT} as the template to generate the upstream fragment of the mutation site with TF-F as the forward primer and PRD25N-R (ATTTAATAGAGCTTC CTTTAATTTGC, SEQ ID NO: 5) as the reverse primer. The second PCR generated the downstream fragment of the mutation site with PRD25N-F (GCAATTAAAGGAAGCTCTATTAAAT, SEQ ID NO: 6) as the forward primer and PR-R as the reverse primer. The changed nucleotides are shown in boldface type, and the codons corresponding to the mutated residues are underlined. The upstream fragment from the first PCR and the downstream fragment from the second PCR were mixed and then used as templates for the final PCR. The final PCRs yielded either PR^{D25N} if PR-F and PR-R were used as primers or TF-PR^{D25N} if TF-F and PR-R were used. The amplified fragments were then digested with NcoI and subcloned into pβ-Gal^{PR}. The resulting construct was designated pPR^{D25N} or pTF-PR^{D25N}, respectively.

The HIV protease precursor variant containing the D30N mutation or the I84V mutation was similarly constructed by PCR-mediated mutagenesis as described previously (Ansaldi et al., supra). The oligonucleotides TF-F and PRD30N-R (CCTGGCAAATTCATTTCTTCTAATACTGTGTT, SEQ ID NO:7) or PRI84V-R (TA[C/T]GTTGACAGGTCTAGGTCCTACTAATACTGTACC, SEQ ID NO:8) were used as primers for the first-round PCR to generate the upstream fragment ofthe mutation site. The oligonucleotides PRD30N-F (AACACAGTATTAGAAGAAATGAATTTGCCAGG, SEQ ID NO: 9) or PRI84V-F (GGTACAGTATTAGTAGGACCTACACCTGTCAAC[A/G]TA, SEQ ID NO: 10) and PR-R were used as primers for the second-round PCR to generate the downstream fragment containing the mutationisite. The corresponding fragment pairs were then combined, and TF-F and PR-R were used as primers to amplify the final product, that is, TF-PR^{D30N} or TF-PR^{1184V}. Again, these fragments were digested with NcoI and subdloned into pβ-Gal^{PR}. The resulting plasmids were designated pTF-PR^{D30N} or pTF-PR^{I84V}, respectively. The sequences of all constructs were confirmed by automatic DNA sequencing (DAVIS Sequencing, Davis, Calif.).

### EXAMPLE 4

### Expression of HIV Protease, Mutant HIV Protease, and B-GAL^{PR}

The expression of recombinant proteins in the E. coli cells bearing the expression plasmid, pTF-PR^{WT} or pTF-PR^{D25N}, was analyzed by Western blotting with HIV-1 protease antiserum and with β-galactosidase antibodies. This Example also describes the effect on activity of the engineered β-galactosidase when it is cleaved by the HIV protease.

E. coli cells bearing the respective plasmid, pTF-PR^{WT} or PTF-PR^{mutant}, were inoculated into 2 ml of Luria-Bertani medium with 100 μg of ampicillin/ml and grown overnight at 37° C. The cultures were diluted 100-fold with fresh Luria-Bertani medium containing 100 μg of ampicillin/ml and incubated at 37° C. for 2 h until the optical density at 630 nm (OD₆₃₀) of the cells reached 0.6. The cells were then induced for protein expression with 0.2% arabinose. After incubation for an additional 3 h at 30° C., the cells were collected by centrifugation and solubilized in Laemmli sample buffer (Laemmli, Nature 227:680-85 (1970)) at a concentration of 0.01 OD₆₃₀ cells per μl of sample buffer. The cellular proteins were separated on 4 to 12% Novex Bis-Tris polyacrylamide gels and electrotransferred onto polyvinylidene fluoride (PVDF) membranes for analysis with specific antibodies. The membranes were blocked with 5% nonfat milk in TTBS (10 mM Tris HCl, pH 7.4, 500 mM NaCl, 0.1% Tween 20) for 1 h and then incubated with 1:5,000 anti-β-galactosidase monoclonal antibodies or 1:5,000 HIV-1 protease antiserum in 1% nonfat milk in TTBS for another hour. The bound antibodies were detected by chemiluminescence using horseradish peroxidase and Lumi-Glo chemiluminescence reagent.

The results are presented in FIGS. 1B and 1C. The immunoblots confirmed that both HIV protease and β-galactosidase could be translated from a single mRNA in this two-cistron system. Most of the wild-type HIV protease precursor was autoprocessed, and the mature HIV protease was released in the E. coli cells (FIG. 1B, lane 3). The mature HIV protease subsequently cleaved β-Gal^{PR}, resulting in the appearance of an extra band with a lower molecular weight in the lysates from the cells expressing TF-PR^{WT} and β-Gal^{PR} (FIG. 1C).

Cell-Based β-Galactosidase Activity Assay.

E. coli cells bearing each of the expression plasmids were grown as described above at 37° C. for 2 h to an OD₆₃₀ of 0.6. The cells were induced for recombinant protein expression. After incubation for an additional 3 hours at 30° C., the cells were made permeable for the entry of the ^{β}-galactosidase substrate, (ortho-nitrophenyl-β-D-galactopyranoside (ONPG)), by diluting the cells in assay buffer (10 mM NaP, pH 7.3, 10 mM NaCl, 1 mM MgSO₄, 5 mM β-mercaptoethanol) that contained 50 μg of polymyxin B sulfate/ml and 2% Triton X-100 (Schupp et al., BioTechniques 19:18-20 (1995)). The cells were then incubated for 5 min at room temperature in 96-well microplates. After the OD₆₃₀ of the cells was determined by using an Ultramark microplate imaging system (Bio-Rad, Hercules, Calif.), ONPG was added at a final concentration of 200 μM to start the enzymatic reactions at 37° C. Changes in the OD₄₁₅, indicating the production of ortho-nitrophenol, were continuously monitored with the Ultramark microplate imaging system. The linear portion of the progression curve was used to determine the initial velocity with MATLAB (The MathWorks Inc., Natick, Mass.). The difference in OD₄₁₅ (ΔA) was converted to the increase in product concentration (ΔC) by using the equation ΔA=ε(ΔC)(L), where L is the light path of 0.4 cm and the extinction coefficient (ε) of ortho-nitrophenol was 3,500 M⁻¹ cm⁻¹ under the assay conditions; The β-galactosidase activity was determined as the number of micromoles of ortho-nitrophenol produced per minute at 37° C. The β-galactosidase activity was then normalized against the amount of total cellular proteins, which was estimated by assuming that an OD₆₃₀ unit corresponds to 1.4×10⁹ cells and that every 10⁹ cells yield approximately 150 μg of proteins (Pardee et al., J. Mol. Biol. 1:165-68 (1959)).

Uninduced E. coli contained no detectable β-galactosidase activity and the E. coli cells expressing only β-Gal^{PR} gave a β-galactosidase activity of 0.191±0.005 μmol/min/mg of total E. coli proteins. When β-galactosidase was co-expressed with HIV protease, β-galactosidase activity was 0.041±0.003 and 0.178±0.007 μmol/min/mg for E. coli producing active and inactive HIV protease, respectively. These data confirmed E. coli β-galactosidase as a valid reporter for in vivo HIV protease activity.

### EXAMPLE 5

### Diversity-Oriented Chemical Synthesis of HIV Protease Inhibitors

In this Example, chemical synthesis of a library of compounds derived from APV is described. The library was provided by Dr. Chi-Huey Wong and Dr. Ashraf Brik (Department of Chemistry and the Skaggs Institute for Chemical Biology, The Scripps Research Institute, La Jolla, Calif.).

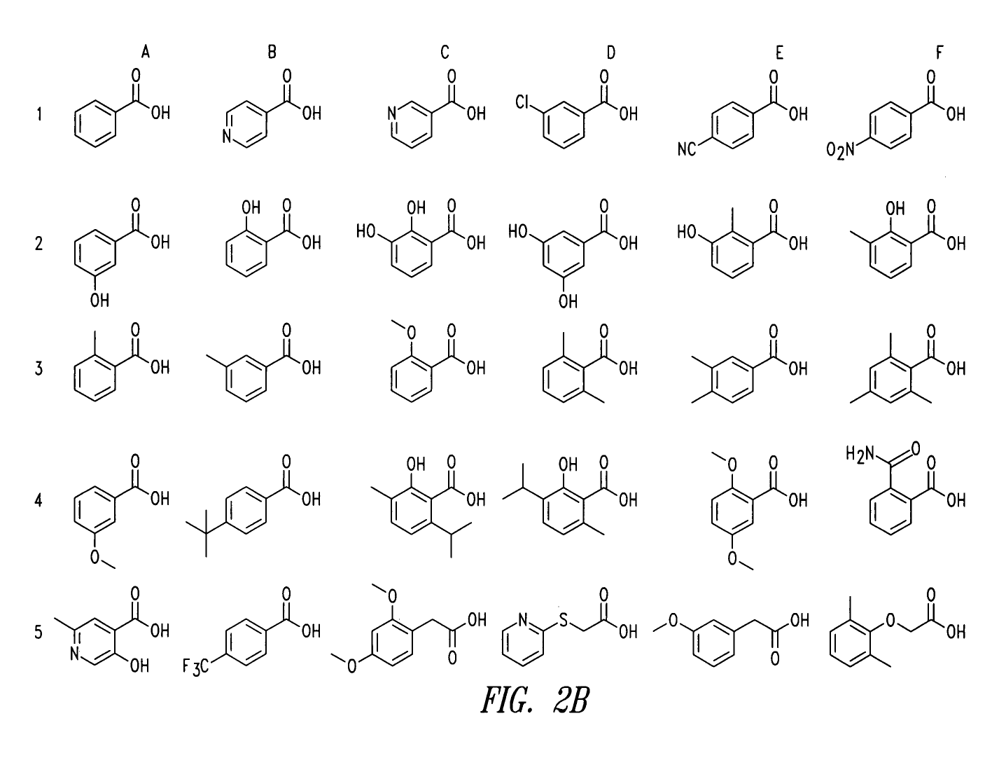

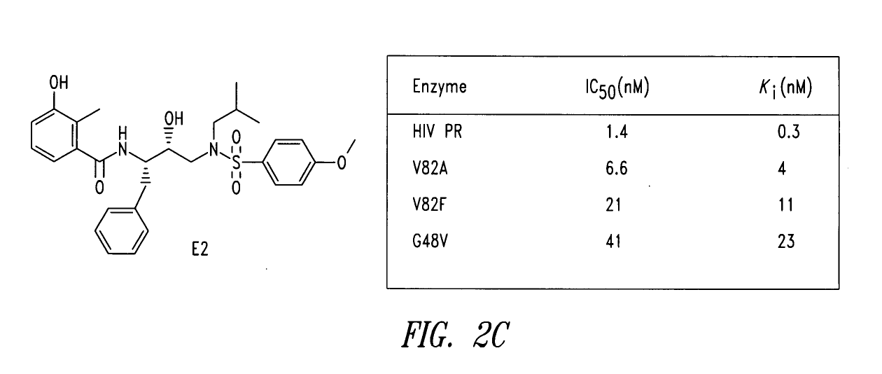

Fifty microliters of each different carboxylic acid (3 μmol) was added to each well of a 96-well microplate that contained 50 μl of N-[(1-H-benzotriazole-1-y) (dimethylamino) methylene]-N-methylmethanaminium hexafluorophosphate N-oxide (HBTU, 3 μmol), and N,N-disopropylethyl amine (DIEA, 6 μmol). As illustrated in FIG. 2A, to each reaction mixture, 50 μl of 2 μmol of amine core 1 dissolved in anhydrous dimethyl formamide (DMF) was added. All the reaction mixtures were mixed and kept at room temperature. The reactions went to completion in 1 hour, based on the disappearance of the free amine, which was monitored by thin-layer. chromatography (10:1 CHCl₃/MeOH ratio; R_{f}=0.28), and analysis of the crude reaction mixture by electrospray ionization mass spectrometry (Brik et al., Chem. Biol. 9:891-96 (2003)).

### EXAMPLE 6

### Synthesis and Purification of Inhibitor E2

In this Example, chemical synthesis of the compound having the structure E2 is described.

Core 1 (see FIG. 2A) was prepared as previously reported (Tung et al., Infect. Dis. Ther. 25:101-18 (2002)). To a solution of a free amine 1 (200 mg, 0.45 mmol), 2-methyl-3-hydroxy benzoic acid (102 mg, 0.67 mmol) in 6 ml of dry DMF was added to HBTU (253 mg, 0.67 mmol) followed by addition of DIEA (230 μl, 1.3 mmol) at 20° C. in an argon atmosphere. The reaction mixture was stirred for 3 h, then quenched by the addition of brine, and extracted with ethyl acetate. The organic layer was washed with 1 N HCl, saturated aqueous NaHCO₃, and brine, dried over MgSO₄, filtered, and concentrated in vacuo. The crude product was purified by flash chromatography (CHCl₃-MeOH), and the desired product was obtained in a 90% yield. The chemical structure of E2 was determined by nuclear magnetic resonance and electrospray ionization mass spectrometry (Brik et al., supra).

### EXAMPLE 7

### Determination of In Vitro IC₅₀S of Inhibitors Against HIV Protease

In this Example, identification of compounds that inhibited HIV protease activity is described.

Recombinant HIV protease for enzymatic assays was prepared as described previously (Brik et al., supra). The determination of kinetic parameters of HIV protease was performed at 37° C. at pH 5.6 by using an F-2000 fluorescence spectrophotometer (Hitachi) and a Packard fluorescence spectrophotometer (Fusion-Universal microplate analyzer) for the microplate assay. For HIV protease, the Kₘ and Vₘₐₓ values for the fluorogenic peptide substrate 2-aminobenzoyl (Abz)-Thr-Ile-Nle˜Phe-(p-NO₂)-Gln-Arg-NH₂ were determined by measuring the initial rate of hydrolysis at different substrate concentrations (2.5, 5.0, 10, 25, 50, and 100 μM) by monitoring the changes in fluorescence at a 420-nm emission and fitting the obtained data to the Michaelis-Menten equation with the GRAFIT program (version 3.0; Erithacus Software, Surrey, United Kingdom). Assays were run in 0.1 M morpholineethanesulfonic acid (MES) buffer containing 0.2 M NaCl and 1 mM dithiothreitol in a final volume of 200 μl in the wells of microplates. The enzyme concentration (30 μg/ml) that gave an ideal progress curve was used for the assays; The in vitro IC₅₀, the concentration required for 50% inhibition of in vitro HIV protease activity, was determined from the percentage of inhibition rendered by E2 at various concentrations: 0.1, 0.5, 2.0, 4.0, 8.0, 12, and 16 nM. The Kᵢ of the compound E2 was derived from the in vitro IC₅₀ by using the formula for competitive inhibition: Kᵢ=IC₅₀/(1+[S]Kₘ).

### EXAMPLE 8

### Treatment Of E. Coli Cells with HIV Protease Inhibitors

In this Example, the effect of different HIV protease inhibitors on processing of the HIV protease precursor and activity of the reporter polypeptide, β-galactosidase, were determined.

Pepstatin A was dissolved in water at 100 mM. Stock solutions of the FDA-approved HIV protease inhibitors were prepared by solubilizing the drugs in 100% dimethyl sulfoxide (DMSO) at 100 mM. A single colony of the cells bearing the respective plasmid, namely pTF-PR^{WT} or pTF-PR^{mutant}, was inoculated into 2 ml of Luria-Bertani (LB) medium with 100 μg of ampicillin/ml and grown overnight at 37° C. The culture was diluted 100-fold with fresh Luria-Bertani medium containing 100 μg of ampicillin/ml and incubated at 37° C. for 2 h until the optical density at 630 nm (OD₆₃₀) of the cells reached 0.6. The cells were then induced for protein expression with 0.2% arabinose and simultaneously treated with either HIV protease inhibitors dissolved in 2% DMSO at the indicated concentrations or 2% DMSO alone as the negative control. After incubation for an additional 3 h at 30° C., the cells were collected for Western blot analysis of proteins.

Western Blot Analysis.

After the cells were induced and treated as described above, they were collected by centrifugation and solubilized in Laemmli sample buffer (Laemmli, Nature 227:680-85 (1970)) at a concentration of 0.01 OD₆₃₀ cells per μl of sample buffer. The cellular proteins were separated on 4 to 12% Novex Bis-Tris polyaciylamide gels and electrotransferred onto polyvinylidene fluoride membranes for analysis with specific antibodies. The membranes were blocked with 5% nonfat milk in TTBS (10 mM Tris HCl, pH 7.4, 500 mM NaCl, 0.1% Tween 20) for 1 h and then incubated with 1:5,000 anti-β-galactosidase monoclonal antibodies or 1:5,000 HIV-1 protease antiserum in 1% nonfat milk in TTBS for another hour. The bound antibodies were detected by chemiluminescence using horseradish peroxidase and Lumi-Glo chemiluminescence reagent.

Cell-Based β-galactosidase Activity Assay. Cell-based β-galactosidase activity assays were performed basically as described in Example 4. E. coli cells bearing each of the expression plasmids were grown as described, except that after incubation at 37° C. for 2 h to an OD₆₃₀ of 0.6, the cells were transferred in triplicate sets to the wells of 96-well microplates. The cells were subjected to simultaneous induction for recombinant protein expression as well as drug treatment with HIV protease inhibitors at the indicated concentrations in 2% DMSO. For the screening of the compound library generated by the amide-forming reaction (see Example 5), DMF was used instead of DMSO. After incubation for an additional 3 h at 30° C., the cells were made permeable for the entry of the β-galactosidase substrate, (ortho-nitrophenyl-β-D-galactopyranoside (ONPG)), by diluting the cells in assay buffer (10 mM NaP, pH 7.3, 10 mM NaCl, 1 mM MgSO₄, 5 mM β-mercaptoethanol) that contained 50 μg of polymyxin B sulfate/ml and 2%Triton X-100 (Schupp et al., BioTechniques 19:18-20 (1995)). The cells were then incubated for 5 min at room temperature in 96-well microplates. After the OD₆₃₀ of the cells was determined by using an Ultramark microplate imaging system (Bio-Rad, Hercules, Calif.), ONPG was added at a final concentration of 200 μM to start the enzymatic reactions at 37° C. Changes in the OD₄₁₅, indicating the production of ortho-nitrophenol, were continuously monitored with the Ultramark microplate imaging system. The linear portion of the progression curve was used to determine the initial velocity with MATLAB (The MathWorks Inc., Natick, Mass.). The difference in OD₄₁₅ (ΔA) was converted to the increase in product concentration (ΔC) by using the equation ΔA=ε(ΔC)(L), where L is the light path of 0.4 cm and the extinction coefficient (ε) of ortho-nitrophenol was 3,500 M⁻¹ cm⁻¹ under the assay conditions. The β-galactosidase activity was determined as the number of micromoles of ortho-nitrophenol produced per minute at 37° C. The β-galactosidase activity was then normalized against the amount of total cellular proteins, which was estimated by assuming that an OD₆₃₀ unit corresponds to 1.4×10⁹ cells and that every 10⁹ cells yield approximately 150 μg of proteins (Pardee et al., J. Mol. Biol. 1:165-68 (1959)).

The in vivo inhibitory effects of the HIV protease inhibitors were evaluated by a peptide-based aspartic protease inhibitor, pepstatin A, and the FDA-approved small-molecule HIV protease inhibitors APV, IDV, NFV, RTV, and SQV. Drug permeability was evaluated by Western blot analysis in which E. coli cells were treated with HIV protease inhibitors without the use of permeabilization agents. The E. coli cells containing the expression construct pPR^{D25N}, pTF-PR^{WT}, or pTF-PR^{D25N} were induced with 0.2% arabinose for 3 hours at 30° C. in the presence of 2% DMSO as described above. Simultaneously, the E. coli cells expressing TF-PR^{WT} were treated with the HIV protease inhibitors at the indicated concentrations in 2% DMSO. The cells were then collected, and the proteins were analyzed by Western blotting with HIV-1 antiserum. Each lane contained 0.3 OD₆₃₀ equivalents of cells, except the lane for PR^{D25N}, which contained 0.1 OD₆₃₀ equivalents of cells expressing PR^{D25N}.

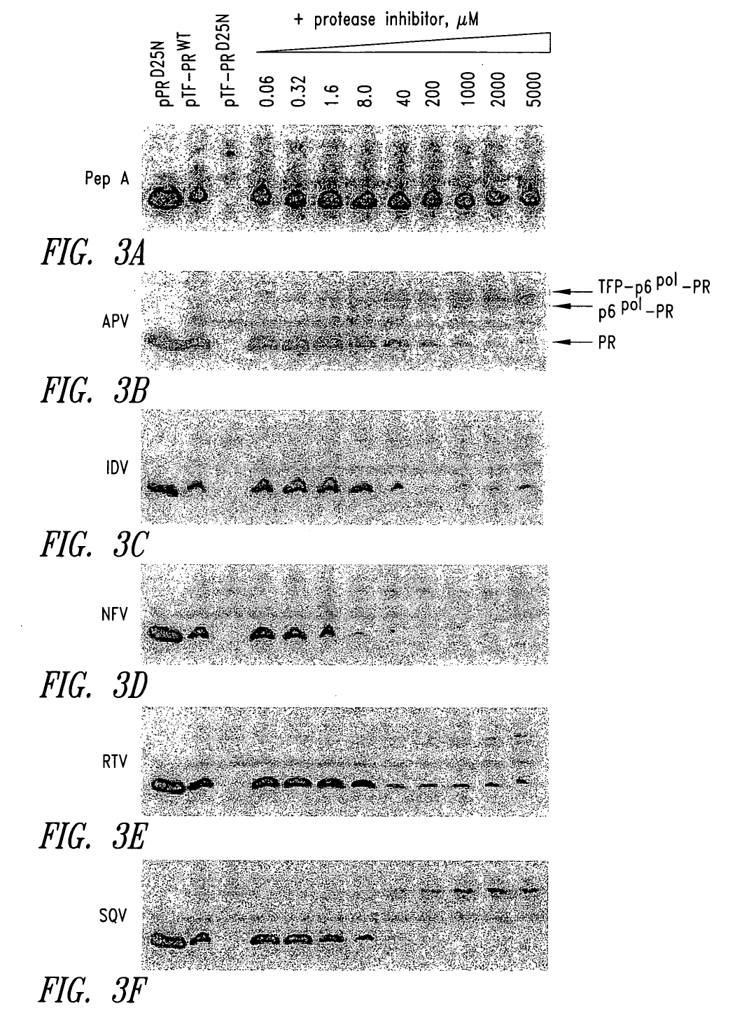

As shown in FIG. 3, all small-molecule HIV protease inhibitors, except pepstatin A, inhibited the autoprocessing of the HIV protease precursor in a dose-dependent fashion. The dose-dependent inhibition ofautoprocessing could be detected with 1.6 μM HIV protease inhibitors and reached a limit with 200 M HIV protease inhibitors. The cleavage at the TFP-p6^{pol} site, resulting in the release of p6^{pol}-PR, still occurred inthe presence of 5 mM HIV protease inhibitors (FIG. 3). The concentration of IDV and RTV needed for 50% inhibition of the release of mature HIV protease was between 8 and 40 μM, and the concentrations of APV, NFV, and SQV needed for 50% inhibition of the release of mature HIV protease was between 1.6 and 8 μM.

### EXAMPLE 9

### Dose Dependent Effects of The HIV Protease Inhibitors on β-Galactosidase Activity

In this Example, the potency of HIV protease inhibitors was analyzed by determining the cleavage-induced loss of β-galactosidase activity.

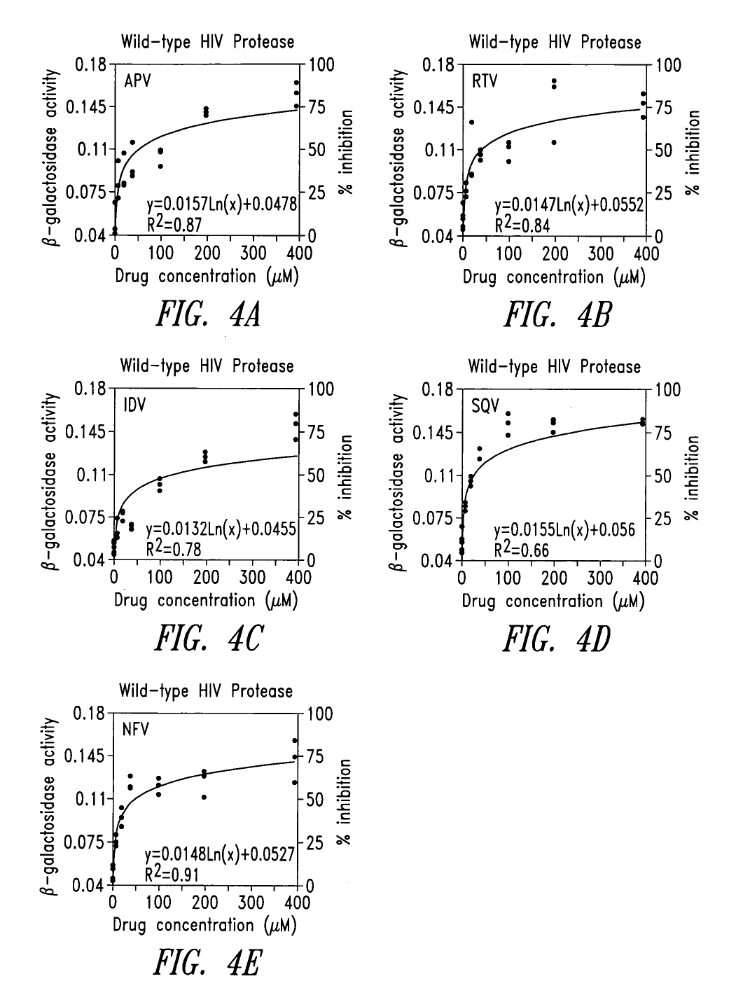

ββ-galactosidase activity assays were performed as described in Example 8. After drug treatment for 3 h, the cells were processed for detection of β-galactosidase activity of whole cells in 96-well microplates as described. Cells coexpressing TF-PR^{WT} and β-Gal_{PR} were treated with HIV protease inhibitors at the concentrations between 0 and 400 μM. β-galactosidase activities (micromoles per minute per milligram of total protein) in the treated E. coli cells were determined. Representative results from three separate experiments are presented in FIG. 4, and each experiment had a triplicate set of each sample.

As shown in FIG. 4, P-galactosidase activity increased exponentially as the concentration of HIV protease inhibitors increased. Consistent with the inhibition of autoprocessing of the HIV protease precursor, the increase in β-galactosidase activity could be detected using 1.6 μM HIV protease inhibitors with minimal variation between 200 μM and 400 μM HIV protease inhibitors, except with IDV. At a concentration of 400 μM HIV protease inhibitors, β-galactosidase activity was approximately 0.145 μmol/min/mg of total proteins, which was a little lower than the maximal β-galactosidase activity detected in the presence of inactive HIV protease. Permeabilization reagents were used to allow the entry of ONPG for the in vivo β-galactosidase assay and added only after cells were harvested after drug treatment. Thus, the permeabilization reagents did not affect the permeability of HIV protease inhibitors.

Pepstatin A did not inhibit β-galactosidase activity. Pepstatin A has an in vitro IC₅₀ of 0.7 μM against the purified HIV protease (Krausslich et al., Proc. Nat'l Acad. Sci. USA 86:807-11 (1989)). As determined according to the assay procedures described herein, pepstatin A, up to 5 mM, failed to exert any inhibitory effects. These data indicated that pepstatin A has low cell permeability; thus, it fails to diffuse passively across the cell membrane to inhibit intracellular autoprocessing of the HIV protease precursor.

### EXAMPLE 10

### IC50 of The HIV Protease Inhibitors Against Wild Type HIV Protease Activity

In this Example, the IC₅₀ of a given HIV protease inhibitor against the wild-type HIV protease in the E. coli system was determined by curve-fitting with MATLAB (FIG. 4; see also Table 1 in Example 11). IC₅₀ refers to the concentration of the HIV protease inhibitor required to reach a 50% inhibition of the cleavage-induced loss of β-galactosidase activity. The β-galactosidase activity detected in E. coli coexpressing TF-PR^{WT} and β-Gal^{PR} in the absence of HIV protease inhibitors was referred to as 0% inhibition. The maximal β-galactosidase activity observed in E. coli containing pTF-PR^{D25N} was regarded as 100% inhibition, which could theoretically be achieved by the most potent HIV protease inhibitor. As shown in Table 1, the IC₅₀ for each HIV protease inhibitor was in the micromolar range, which is higher. than values determined in standard antiviral assays.

### EXAMPLE 11

### IC₅₀ of The HIV Protease Inhibitors Against Mutant HIV Protease Activity

In this Example, the usefulness of the E. coli-based assay system described herein is demonstrated using a D30N HIV protease mutant, a variant observed in HIV clinical isolates that specifically confers drug resistance to NFV. The D30N mutation is an active-site mutation and represents the major mutation that confers drug resistance specifically against NFV. The structural analysis of binary complex D30N HIV protease with NFV indicated altered interactions with hydrophobic P2 side chains, resulting in variable substrate specificity and lower catalytic efficiency (Mahalingam et al., Proteins 43:455-64 (2001)). The k_{cat}/Kₘ of D30N HIV protease was determined to be 0.089 min⁻¹ μM⁻¹ compared to 0.41 min^{-1 μM}⁻¹ for the wild-type HIV protease (Mahalingam et al., Eur. J. Biochem. 263:238-45 (1999)). This indicated that the D30N HIV protease has a catalytic efficiency fivefold lower than the wild-type HIV protease.

The HIV protease D3ON mutant was prepared by PCR-mediated mutagenesis as described in Example 3 and was used to replace the wild-type HIV protease domain in the two-cistron expression construct for co-expression together with β-galactosidase to create the construct pTF-PR^{D30N}. After E. coli cells that contained the expression plasmid pTF-PR^{D30N} were induced to express recombinant protein, β-galactosidase activity was determined using whole cells in 96-well microplates (see Example 4). The β-galactosidase activity in the E. coli cells co-expressing TF-PR^{D30N} and β-Gal^{PR} was 0.074±0.011 μmol/min/mg of total proteins, which was higher than the β-galactosidase activity (0.041±0.003 μmol/min/mg of total proteins) detected in the presence of the wild-type HIV protease. This indicated that the D30N HIV protease mutant has a lower catalytic efficiency upon cleaving the substrate β-galactosidase, resulting in observation of an increase in β-galactosidase activity compared with β-galactosidase activity determined in the presence of the wild-type HIV protease. Therefore, this system is suitable for detecting HIV protease variants that may have differing levels of activities from the wild-type HIV protease.

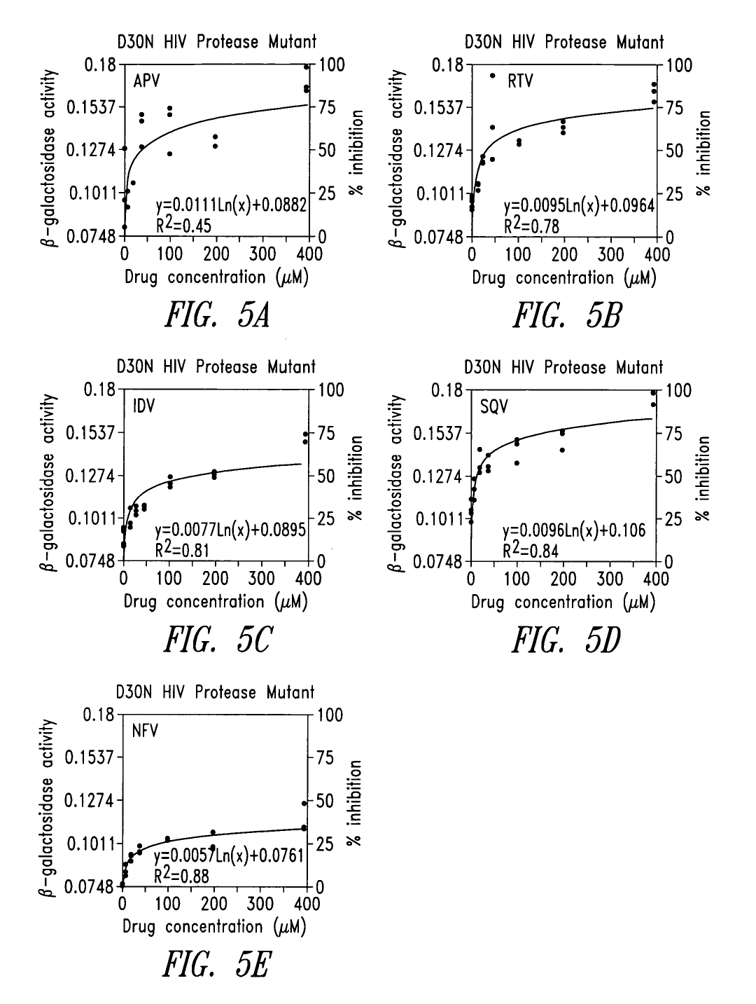

The drug response of the D30N HIV protease to HIV protease inhibitors was investigated by using an experimental protocol similar to that used in the study of the wild-type HIV protease. β-galactosidase activities (micromoles per minute per milligram of total protein) in the treated E. coli cells were determined, and the data were used to generate the dose-response curves by curve-fitting with MATLAB. Representative results from three separate experiments are shown, and each experiment had a triplicate set of each sample. The results are presented in FIG. 5. In this study, 0% inhibition was defined as the β-galactosidase activity detected in theE. coli cells co-expressing the D30N HIV protease and β-Gal^{PR} in the absence of HIV protease inhibitors. Reference to 100% inhibition was the same as discussed in Example 10. As shown in FIG. 5, the sensitivities of the D30N mutant HIV protease toward APV, IDV, RTV, and SQV were similar to those for the wild-type HIV protease. 400 μM NFV exhibited 30% inhibition against the D30N HIV protease compared to 75% inhibition against the wild-type HIV protease. The IC₅₀ of NFV for the D30N HIV protease mutant was thus determined to be greater than 400 μM, at least nine-fold higher than the IC₅₀ determined for the wild-type HIV protease (see Table 1).

TABLE 1
Comparison of IC₅₀s of protease inhibitors determined using three methods

| | IC₅₀ (μM): E. coli-based | | EC₉₀ (μM) from standard | | |
|---|---|---|---|---|---|
| HIV | assay system^{a} with: | | antiviral assay^{b} with: | | IC₅₀ (μM): E. coli-based |
| protease | Wild-type | D30N HIV | Wild- | D30N | assay using wild-type HIV |
| inhibitor | HIV protease | protease | type HIV | HIV strain | protease^{c} (Dautin et al.) |
| APV | 53.4 ± 17.4 | 35.9 ± 17.7 | —^{e} | — | — |
| IDV | 132.5 ± 17.5 | 137.3 ± 13.7 | 0.060 | 0.010 | 100 |
| NFV | 48.4 ± 4.8 | >400.0 | 0.030 | 0.180 | — |
| RTV | 42.6 ± 9.1 | 26.9 ± 7.5 | 0.050 | 0.010 | — |
| SQV | 23.7 ± 3.4 | 9.3 ± 3.5 | 0.030 | 0.010 | 20 |
| Pepstatin A | ND^{d} | ND | — | — | — |

^{a}IC₅₀ refers to the inhibitor concentration required to reach a 50% inhibition of the loss of β-galactosidase activity resulting from HIV protease-induced cleavage. IC₅₀s and standard errors were determined from three separate experiments as shown in FIGS. 4 and 5.
^{b}See Patick et al., Antimicrob. Agents Chemother. 42: 2637-44 (1998). EC₉₀ refers to the drug concentration that inhibited 90% of mammalian cell death induced by HIV infection.
^{c}See Dautin et al., J. Bacteriol 182: 7060-66 (1992). IC₅₀ refers to the inhibitor concentration required to reach a 50% inhibition of the cyclic AMP production induced by HIV protease.
^{d}ND, not detectable.
^{e}—, not reported in literature

### EXAMPLE 12

### Identification of HIV Protease Activity Inhibitors

In this Example, the compound library prepared in Example 5 was screened in situ against the HIV protease. The compounds in the library were initially screened at a concentration of 100 nM. The compounds that exhibited 50% or greater inhibition were selected for a second screening at 10 nM. The two rounds of screening revealed the preference of aromatic residues at the P2 position, such as benzoic acid. More than thirty acids of ortho-, meta-, para-substituted benzoic acids (FIG. 2B) were coupled to the core and screened at 5 nM. Compound E2 emerged as the best inhibitor from this library (Table 2). All measurements were run in triplicate, and the reported values are the means of these measurements.

TABLE 2
Percentage of in vitro HIV protease activity detected in
the presence of 5 nM inhibitors from the compound library

| | HIV protease activity |
|---|---|
| R-COOH | (%) with 5 nM inhibitor |
| A1 | 75 |
| B1 | 70 |
| C1 | 75 |
| D1 | 70 |
| E1 | 68 |
| F1 | 65 |
| A2 | 35 |
| B2 | 74 |
| C2 | 80 |
| D2 | 50 |
| E2 | 0 |
| F2 | 90 |
| A3 | 5 |
| B3 | 80 |
| C3 | 96 |
| D3 | 30 |
| E3 | 60 |
| F3 | 60 |
| A4 | 90 |
| B4 | 94 |
| C4 | 99 |
| D4 | 98 |
| E4 | 93 |
| F4 | 95 |
| A5 | 90 |
| B5 | 70 |
| C5 | 97 |
| D5 | 86 |
| E5 | 79 |
| F5 | 40 |

E2 was synthesized and purified as described in Example 6. The in vitro IC₅₀s and Kᵢ values of the purified E2 against the wild-type HIV protease and three other HIV protease mutants were subsequently determined. The results are presented in FIG. 2C.

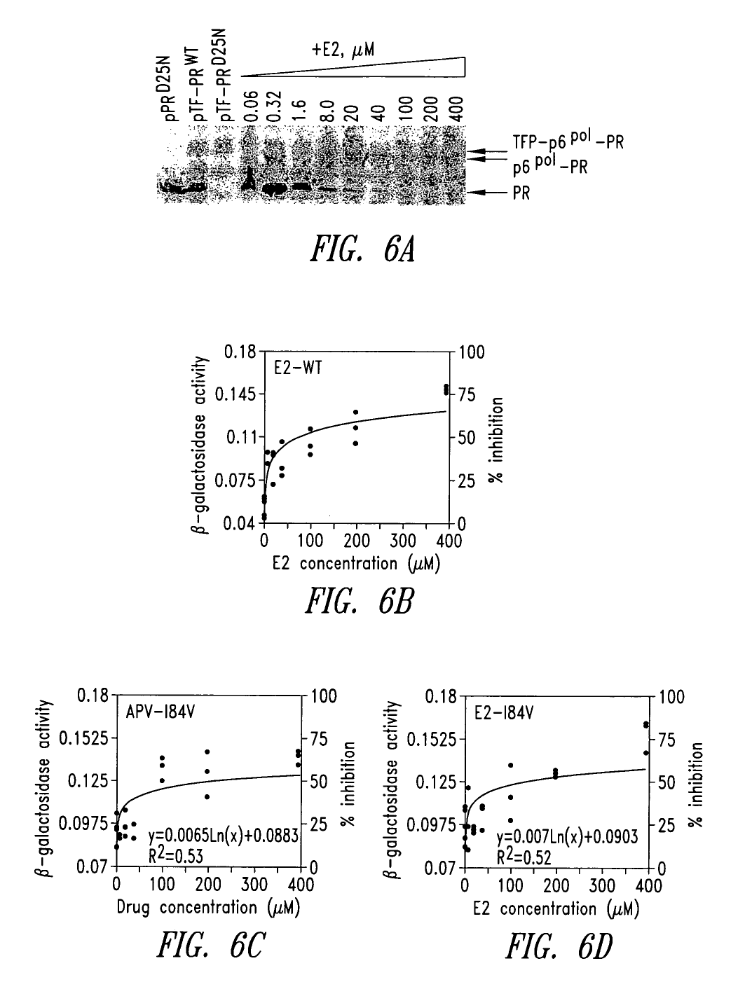

The compounds in the library were also tested for inhibition of HIV protease activity in the E. coli-based model system. The concentration of each compound in the initial screening was 200 μM. Consistent with in vitro enzymatic studies, E2 was identified as the most potent compound in the E. coli-based model system. As shown in FIG. 6A, E2 at 1.6 μM inhibited autoprocessing of the HIV protease precursor, and inhibition was maximum at 100 μM E2. β-galactosidase activity in the cells treated was determined using different concentrations of E2. The in vivo IC₅₀ of E2 in the E. coli-based system was 87.3±25.4 μM, which is comparable to the potency of drugs currently used to inhibit HIV protease (see FIG. 6B and Table 1).

### EXAMPLE 13

### Activity of HIV Protease Inhibitors Against Mutant HIV Proteases

Because the core of inhibitor E2 is similar to that of APV, the drug response of E2 against the APV-resistant HIV protease mutants was determined. One key signature substitution in the HIV protease that results in APV resistance is substitution of isoleucine at position 84 with valine (I84V) (Gong et al., Antimicrob. Agents Chemother. 44:2319-26 (2000)). The in vivo EC₉₀ (inhibitor concentration that inhibits 90% of virus-induced cytotoxicity) of APV against 184V HIV protease mutant is twofold higher than the EC₉₀ against the wild-type HIV protease (Klabe et al., Biochemistry 37:8735-42 (1998)). The in vitro enzymatic assay also showed a threefold increase of the Kᵢ of APV against the 184V HIV protease mutant compared to that of the wild-type HIV protease (Klabe et al., supra).

The I84V HIV protease mutant was prepared by PCR-mediated mutagenesis and used to replace the wild-type HIV protease domain in the two-cistron system to construct the plasmid pTF-PR^{I84V} (see Example 3). After inducing E. coli cells bearing the expression plasmid pTF-PR^{I84V}, β-galactosidase activity was determined (see Example 4). The β-galactosidase activity in the cells expressing TF-PR^{I84V} and β-Gal^{PR} was 0.081±0.001 μmol/min/mg of total proteins, which was higher than the β-galactosidase activity (0.041±0.003 μmol/min/mg of total proteins) detected in the presence of the wild-type HIV protease. This result-confirmed that the I84V HIV protease mutant had a lower catalytic efficiency than the wild-type HIV protease in this E. coli-based system.

The drug response of the I84V HIV protease mutant toward E2 was investigated by using similar experimental protocols. and included APV as a control. The in vivo activity of the HIV protease inhibitors was determined by the inhibition of the cleavage-induced loss of β-galactosidase activity. The cells coexpressing TF-PR^{I84V} and β-Gal^{PR} were treated with HIV protease inhibitors at the concentrations indicated in FIG. 6C and FIG. 6B. β-Galactosidase activities (micromoles per minute per milligram of total protein) in the treated E. coli cells were determined, and the data were used to generate the dose-response curves by curve-fitting with MATLAB. Representative results from three separate experiments are shown, and each experiment had a triplicate set of each sample. In this study, 0% inhibition was defined as the β-galactosidase activity detected in E. coli co-expressing the I84V HIV protease and β-Gal^{PR} without treatment with HIV protease inhibitors. The IC₅₀ of E2 against I84V HIV protease was 141.3±50 μM while the IC₅₀ of APV was 280±39 μM (see FIGS. 6C and 6D).

From the foregoing, those skilled in the art will recognize or be able to ascertain, using no more than routine experimentation, many equivalents to the specific embodiments of the invention described herein. Such equivalents are intended to be encompassed by the following claims.

1. A polynucleotide comprising a nucleotide sequence that encodes a precursor of a protease and a reporter polypeptide, said reporter polypeptide comprising a protease recognition sequence.

2. The polynucleotide according to claim 1 wherein the precursor of the protease comprises a human immunodeficiency virus (HIV) transframe region and the protease.

3. The polynucleotide according to claim 1 wherein the precursor of the protease is autoproteolytically processed to yield the protease.

4. The polynucleotide according to claim 1 wherein the protease is an aspartyl protease.

5. The polynucleotide according to claim 1 wherein the protease is a viral protease.

6. The polynucleotide according to claim 5 wherein the viral protease is a human immunodeficiency virus (HIV) protease.

7. The polynucleotide according to claim 1 wherein the precursor of the protease comprises a human immunodeficiency virus (HIV) transframe region, and wherein the protease is an HIV protease.

8. The polynucleotide according to claim 1 wherein the reporter polypeptide is β-galactosidase.

9. The polynucleotide according to claim 1 wherein the encoded protease recognition sequence comprises the amino acid sequence VSFNFPQITL (SEQ ID NO: 1).

10. The polynucleotide according to claim I wherein the polynucleotide is a 2-cistron construct, wherein a first cistron encodes the precursor of a protease and a second cistron encodes the reporter polypeptide.

11. The polynucleotide according to claim 1 wherein the protease recognition sequence is inserted at a position in the reporter polypeptide such that in the absence of cleavage of the reporter polypeptide by the protease, the reporter polypeptide has reporter activity that is comparable to reporter activity of the wild type reporter polypeptide, and such that in the presence of cleavage by the protease, the reporter polypeptide has decreased reporter activity compared with the wild type reporter polypeptide.

12. A recombinant expression vector comprising at least one promoter operatively linked to the polynucleotide of claim 1.

13. The recombinant expression vector according to claim 12 wherein the precursor of the protease comprises a human immunodeficiency virus (HIV) transframe region and the protease.

14. The recombinant expression vector according to claim 12 wherein the precursor of the protease is autoproteolytically processed to yield the protease.

15. The recombinant expression vector according to claim 12 wherein the protease is a viral protease.

16. The recombinant expression vector according to claim 15 wherein the viral protease is a human immunodeficiency virus (HIV) protease.

17. The recombinant expression vector according to claim 12 wherein the reporter polypeptide is P-galactosidase.

18. The recombinant expression vector according to claim 12 wherein the encoded protease recognition sequence comprises the amino acid sequence VSFNFPQITL (SEQ ID NO: 1).

19. The recombinant expression vector according to claim 12 wherein the polynucleotide is a 2-cistron construct, wherein a first cistron encodes the precursor of a protease and a second cistron encodes the reporter polypeptide.

20. A host cell comprising the recombinant expression vector according to claim 12.

21. The host cell according to claim 20 wherein the host cell is a bacterial cell.

22. The host cell according to claim 20 wherein the bacterial cell is an E. coli cell.

23. A method for identifying an agent that inhibits catalytic activity of a protease comprising:
(a) contacting a candidate agent with a cell that expresses a precursor of a protease and that expresses a reporter polypeptide, wherein the reporter polypeptide comprises a protease recognition sequence, under conditions and for a time that permit expression and autoproteolytic processing of the precursor of the protease and cleavage of the reporter polypeptide by the protease;
(b) detecting a reporter activity of the reporter polypeptide; and
(c) comparing a level of reporter activity of the reporter polypeptide in the presence and absence of the candidate agent, wherein an increase in the level of reporter activity of the reporter polypeptide in the presence of the candidate agent compared with a level of reporter activity of the reporter polypeptide in the absence of the candidate agent indicates that the candidate agent inhibits the protease.

24. The method of claim 23 wherein the protease is a protease from an infectious disease organism and the reporter polypeptide is β-galactosidase.

25. A method for identifying an agent that inhibits catalytic activity of a human immunodeficiency virus (HIV) protease comprising:
(a) contacting a candidate agent with a cell that expresses a precursor of an HIV protease and that expresses a reporter polypeptide, wherein the reporter polypeptide comprises an HIV protease recognition sequence, under conditions and for a time that permit expression and autoproteolytic processing of the precursor of the HIV protease and cleavage of the reporter polypeptide by the HIV protease;
(b) detecting a reporter activity of the reporter polypeptide; and
(c) comparing a level of the reporter activity of the reporter polypeptide in the presence and absence of the candidate agent; wherein an increase in the level of the reporter activity of the reporter polypeptide in the presence of the candid ate agent compared with a level of reporter activity of the reporter polypeptide in the absence of the candidate agent indicates that the candidate agent inhibits the HIV protease.

26. The method according to claim 25 wherein the reporter polypeptide is β-galactosidase.

27. The method according to claim 25 wherein the HIV protease recognition sequence comprises the amino acid sequence VSFNFPQITL (SEQ ID NO: 1).

28. The method according to claim 25 wherein the HIV protease is an HIV protease mutant.

29. The method according to claim 25 wherein the precursor of the HIV protease comprises an HIV transframe region.

30. The method according to claim 25 wherein the cell is the host cell according to claim 20.

31. A method for determining sensitivity of a human immunodeficiency virus (HIV) isolate to an inhibitor of HIV protease activity comprising:
(a) preparing a host cell that comprises a recombinant expression vector comprising a promoter operatively linked to (i) a nucleotide sequence that encodes an HIV transframe region fused to the nucleotide sequence encoding an HIV protease from the HIV isolate and (ii) a nucleotide sequence that encodes a reporter polypeptide comprising a protease recognition sequence;
(b) contacting an inhibitor of HIV protease activity with the host cell, under conditions and for a time that permit expression and autoproteolytic processing of the precursor of the HIV protease and cleavage of the reporter polypeptide by the HIV protease;
(c) detecting a reporter activity of the reporter polypeptide; and
(d) comparing a level of reporter activity of the reporter polypeptide in the presence and absence of the inhibitor of HIV protease activity, wherein an alteration in the level of reporter activity of the reporter polypeptide in the presence of the inhibitor of HIV protease activity compared with level of reporter activity of the reporter polypeptide in the absence of the inhibitor of HIV protease activity indicates the level of sensitivity of the HIV protease from the HIV isolate to the inhibitor of HIV protease activity.

32. The method according to claim 31 wherein the HIV isolate is obtained from a biological sample.

33. The method according to claim 32 wherein the biological sample is obtained form a subject who is infected with the HIV isolate.

34. The method according to claim 31 wherein the reporter polypeptide is β-galactosidase.

35. The method according to claim 31 wherein the encoded protease recognition sequence comprises the amino acid sequence VSFNFPQITL (SEQ ID NO: 1).

36. The method according to claim 31 wherein the inhibitor of HIV protease activity is selected from amprenavir (Agenerase®); fosamprenavir (Lexiva®); indinavir (Crixivan®); nelfinavir (Viracept®); ritonavir (Norvir®); and saquinavir (Fortovase®).

37. An assay system comprising (a) a cell that comprises a recombinant expression vector comprising a promoter operatively linked to (i) a nucleotide sequence that encodes an HIV transframe region fused to the nucleotide sequence that encodes an HIV protease to provide a precursor of the HIV protease and (ii) a nucleotide sequence that encodes a reporter polypeptide comprising an HIV protease recognition sequence; (b) a compound that induces expression of the precursor of the HIV protease and the reporter polypeptide; and (c) a reporter polypeptide substrate.

38. The assay system according to claim 37 wherein the host cell is a bacterial cell.

39. The assay system according to claim 38 wherein the bacterial cell is an E. coli cell.

40. The assay system according to claim 37 wherein the reporter polypeptide is β-galactosidase.

41. The assay system according to claim 37 wherein the reporter polypeptide substrate is o-nitrophenyl-β-D-galactoside (ONPG) or 6,8 difluoro-4-methylumbelliferyl β-d-galactopyranoside.

42. The assay system according to claim 37 wherein the encoded protease recognition sequence comprises the amino acid sequence VSFNFPQITL (SEQ ID NO: 1).

43. The assay system according to claim 37 wherein the system further comprises an agent that inhibits HIV protease activity.

44. The assay system according to claim 37 wherein the system further comprises at least one candidate agent to be screened for its capability to inhibit HIV protease activity.


SEQUENCE LISTING (21 sequences)

SEQ ID NO: 1 - protein, 10 aa, Artificial Sequence
  Protease recognition site
VSFNFPQITL

SEQ ID NO: 2 - DNA, 37 bp, Artificial Sequence
  Forward PCR primer (oligonucleotide PR-F)
ataccatggcccctcagatcactctttggcagcgacc

SEQ ID NO: 3 - DNA, 30 bp, Artificial Sequence
  Reverse PCR primer (oligonucleotide PR-R)
agcccatgggttattcctccttaaaattta

SEQ ID NO: 4 - DNA, 36 bp, Artificial Sequence
  Forward PCR primer  (oligonucleotide TF-F)
cataccatgggcttttttagggaagatctggccttc

SEQ ID NO: 5 - DNA, 26 bp, Artificial Sequence
  Reverse PCR primer (oligonucletodie PRD25N-R)
atttaatagagcttcctttaatttgc

SEQ ID NO: 6 - DNA, 25 bp, Artificial Sequence
  Forward PCR primer (oligonucletodie PRD25N-F)
gcaattaaaggaagctctattaaat

SEQ ID NO: 7 - DNA, 32 bp, Artificial Sequence
  PCR primer (oligonucleotide PRD30N-R)
cctggcaaattcatttcttctaatactgtgtt

SEQ ID NO: 8 - DNA, 36 bp, Artificial Sequence
  PCR primer (oligonucleotide PRI84V-R)
tangttgacaggtctaggtcctactaatactgtacc

SEQ ID NO: 9 - DNA, 32 bp, Artificial Sequence
  PCR primer (oligonucleotide PRD30N-F)
aacacagtattagaagaaatgaatttgccagg

SEQ ID NO: 10 - DNA, 36 bp, Artificial Sequence
  PCR primer (oligonucleotide PRI84V-F)
ggtacagtattagtaggacctacacctgtcaacnta

SEQ ID NO: 11 - protein, 8 aa, Artificial Sequence
  HIV protease recognition sequence
SQNYPIVQ

SEQ ID NO: 12 - protein, 8 aa, Artificial Sequence
  HIV protease recognition sequence
ARVLAEAM

SEQ ID NO: 13 - protein, 8 aa, Artificial Sequence
  HIV protease recognition sequence
ATIMMQRG

SEQ ID NO: 14 - protein, 8 aa, Artificial Sequence
  HIV protease recognition sequence
PGNFLQSR

SEQ ID NO: 15 - protein, 8 aa, Artificial Sequence
  HIV protease recognition sequence
SFNFPQIT

SEQ ID NO: 16 - protein, 8 aa, Artificial Sequence
  HIV protease recognition sequence
TLNFPISP

SEQ ID NO: 17 - protein, 8 aa, Artificial Sequence
  HIV protease recognition sequence
AETFYVDG

SEQ ID NO: 18 - protein, 8 aa, Artificial Sequence
  HIV protease recognition sequence
RKILFLDG

SEQ ID NO: 19 - protein, 8 aa, Artificial Sequence
  HIV protease recognition sequence
QITLWQRP

SEQ ID NO: 20 - protein, 8 aa, Artificial Sequence
  HIV protease recognition sequence
DTVLEEMS

SEQ ID NO: 21 - protein, 8 aa, Artificial Sequence
  HIV protease recognition sequence
DQILIEIC